(19) United States
(12) Patent Application Publication
(10) Pub. No.: US 2010/0173001 A1
(43) Pub. Date: Jul. 8, 2010

(54) Metal Ion-Treated Biocompatible Polymers Useful for Nanoparticles

(75) Inventor: Gretchen M. Unger, Chaska, MN (US)

Correspondence Address: SWANSON & BRATSCHUN, L.L.C., 8210 SOUTHPARK TERRACE, LITTLETON, CO, 80120, US

(73) Assignee: GENESEGUES, INC., Chaska, MN (US)

(21) Appl. No.: 12/664,610

(22) PCT Filed: Jun. 16, 2008
(86) PCT No.: PCT/US08/67158

§ 371 Date: Dec. 14, 2009

Related U.S. Application Data
(60) Provisional application No. 60/944,028, filed on Jun. 14, 2007.

(51) Int. Cl. A61K 9/16; A61K 38/00; A61K 31/7088; C07K 2/00; C07H 1/00; A61P 35/04

(52) U.S. Cl. 424/491; 424/490; 424/493; 514/2; 514/44 R; 514/44 A; 530/300; 536/1.11; 977/773; 977/906

(57) ABSTRACT

Disclosed are methods for forming particles useful for the treatment of hyperproliferative disease. The method includes providing a bioactive component and a metal ion-treated biocompatible polymer component; coating the bioactive component with a surfactant having an HLB value of less than about 6.0 units under conditions which form a coated bioactive component; associating the coated bioactive component with the a metal ion-treated biocompatible polymer under conditions which associate the coated bioactive component with the metal-ion treated biocompatible polymer to form a particle, where the particles have an average diameter of less than about 50 nanometers. Related compositions and methods to treat disease using the particles are also disclosed.

### BACKGROUND OF THE INVENTION

Understanding of cancer genes and cellular mechanisms has improved tremendously over the past three decades, but this has not translated into equivalent benefits to cancer patients. Cases of improved survival mostly reflect early detection or prevention, rather than improved treatment. Many believe that the efficacy of conventional cancer therapies, cytotoxics and radiation, has reached a plateau in the treatment of many cancers.

Armed with better knowledge of cancer genetics, current therapeutic strategies aim to produce drugs that eliminate tumor cells while sparing normal tissues. This targeted approach is aimed specifically at genes whose products are involved in cancer, and that are ‘druggable’. This has produced a few impressive drugs that have revolutionized the treatment of certain cancers, such as rituximab for treatment of non-Hodgkin lymphoma. However, most cancers are extraordinarily heterogenous, involving hundreds of mutated and deregulated genes, and often show either transient benefits or no benefit at all.

With a better understanding of cancer cellular mechanisms, drug delivery strategies such as nanoplexes, lipoplexes, polyplexes, and antibody-drug conjugates are employed to deliver molecular-targeted therapies to specific cell targets, and thereby reduce toxicity. Certain delivery vehicles are also capable of delivering therapeutic cargo into the target cell, increasing the population of “druggable” targets. However, the benefits of these delivery strategies would not be expected to overcome in most cases the therapeutic challenges associated with the heterogeneity of cancer. Thus, there is an urgent sense that new strategies for the treatment of cancer are needed.

### SUMMARY OF THE INVENTION

The present invention affords a means of treating or improving treatment of hyperproliferative disease at primary and disseminated sites. The invention is based at least in part upon the surprising discovery that very low dosages of metals can be bound with polymer ligands of a nanocapsule to improve the antiproliferative effects of the drug-carrying nanocapsule, without compromising ligand targeting function or generating any apparent toxicity.

Disclosed are methods and compositions for treatment, prevention, or reduction of hyperproliferative disease. In one embodiment the method comprises incorporating one or more metal compositions in a nanocapsule, where the metal composition is bound to one or more polymers comprising the shell of the nanocapsule, where the polymer targets abnormally hyperproliferative cells (hence, hyperproliferative disease), including targeting of tumor cells, and where the metal is provided in an amount sufficient to enhance the anti-proliferative activity of the nanocapsule, and wherein the nanocapsule enters the cell and releases the agents that modulate cellular activity. In this embodiment, the nanocapsule also incorporates in the core a drug, such as a nucleic acid, protein, peptide, small molecule, and/or metals. In the event metals are incorporated in the core, they may or may not be the same metal-type as those incorporated in the polymer comprising the shell.

In another example embodiment the method comprises incorporating one or more metal compositions in a nanocapsule, where the metal composition is bound to one or more polymers comprising the shell of the nanocapsule, where the polymer optionally targets abnormally hyperproliferative cells (hence, hyperproliferative disease), including targeting of tumor cells, and where the metal is provided in an amount sufficient to effect or enhance the anti-proliferative activity of the nanocapsule, and wherein the nanocapsule enters the cell and releases the agents that modulate cellular activity. In this embodiment, the nanocapsule also incorporates in the core a nontherapeutic molecule such as a diagnostic and/or visualization agent such as a fluorescent marker or dye. A therapeutic molecule may or may not also be incorporated in the core of this example embodiment.

Metals capable of enhancing or effecting antiproliferative activity in a nanocapsule formulation are also disclosed. In one embodiment, the polymer may be metal-modified with one or more inorganic metal compounds. In another embodiment, the polymer may be metal-modified with one or more organic metal compounds. In another embodiment, the polymer may be metal-modified with both inorganic and organic metal compounds.

The methods and compositions disclosed herein are useful for therapeutic purposes both in vivo and ex vivo, as well as for diagnostic reagents and research reagents, including reagents for the study of both cellular and in vitro events.

All patents and patent applications referenced herein are incorporated by reference in their entireties.

### BRIEF DESCRIPTION OF THE DRAWINGS

FIG. 1 shows a schematic of a method for one embodiment of preparing nanoparticles containing metal-modified biocompatible polymers.

FIG. 2 shows efficacy of particle targeting to primary tumor and metastases. A: Tumor and injection site are primary sites of accumulation tenfibgen-formulated iodine-derivatized model siRNA at 2 hours postinjection by iodine-128 neutron activation analysis (NAA). B-D: Capsules can be detected in NAA tumors and dermal metastases at 2 hr. Mice were treated with 10 mg/kg of s50 iodo-siRNA for testing of NAA as a drug-tracing strategy and euthanized after 2 hours. Cryosections a treated mouse (2B), an untreated mouse (2C) and ulcerated skin showing NAA signal (2D) were double-labeled with goat anti-Syrian Hamster (Jackson, Cy3, B-D) and anti-K14 (Covance, B1-D1) antibodies with ‘blue’ Cy5 secondaries and examined on a Nikon Clsi confocal microscope at x600. Please note that only one thyroid and blood sample was available in the sugar capsule-treated group.

FIG. 3 shows metal pretreatment of coating ligand reduces dosing requirements in vitro. The effect of pretreating nanoparticle coat ligands with diverse metal cocktails on growth inhibition of SCC-15 tongue carcinoma cells was assayed by treating cells with nanoparticles bearing antisense to an antiproliferative target prepared with pretreated ligand using two different schedules. Single and double dosing are accommodated by plotting cumulative dose against cell survival relative to diluent-treated cells. 1500-2000 cells were plated into 96 wells plates. Cells were treated either 24 hour or 24 and 48 hours after plating and cell growth was assayed at 72 hours using the WST (MTT) assay.

FIG. 4 shows change in body weight over time for male SCID mice flank-inoculated with 5×10e6 UM-11A head neck carcinoma cells following chronic intraperitoneal every 3 day dosing of 1) PBS or 10 ug/kg nanoencapsulated (s50) anti-Gapdh (“Controls”, n=3+2), 2) 10 ng/kg s50 anti-CK2 without metal modification (“No Metal”, n=3), 3) 10 ng/kg s50 anti-CK2 with metal modification (“10 ng/kg”, n=6), and 4) 10 ug/kg s50 anti-CK2 with metal modification (“10 ug/kg”, n=6). The line chart shows the running average for treatment group weight over time relative to weight at beginning of treatment.

### DETAILED DESCRIPTION OF THE INVENTION

The present invention provides methods and compositions for treatment of hyperproliferative disease. In particular, the invention provides compositions and methods comprising the use of metals, for effective treatment of hyperproliferative disease. Additionally, the invention provides means for in vitro, in vivo, and ex vivo applications.

The compositions and methods provided herein further expand the utility of metals as tools in researching and treating hyperproliferative disease. This discovery increases the understanding of the use of formulation and targeting strategies with respect to metals, and in view of this discovery, the present invention provides important tools to improve treatment of hyperproliferative diseases.

The present invention is based, at least in part upon the surprising discovery that very low dosages of metals can, in one embodiment of the invention, be noncovalently bound with polymer ligands of a nanocapsule to improve the antiproliferative effects of the nanocapsule, without compromising ligand targeting function or generating any apparent toxicity. The noncovalent binding of metals acts in synergy with the therapeutic agent present in the nanocapsule. The invention is further based, in part, upon the discovery that, at the low dosages contemplated by this invention, multiple metal-types can be bound to the nanocapsule, providing flexibility in targeting and treating the array of hyperproliferative diseases.

Surprisingly, we have found that very low dosages of metals complexed with the polymer ligands of a nanocapsule substantially improve the antitumor effects of the nanocapsule. It is believed at these dosages that many types of metals (particularly heavy metals) would be a feasible candidate for incorporation in the nanocapsule. This is a significant advantage compared with conventional antibody drug conjugates, where typically the number of therapeutic agents that can be linked to an antibody is relatively low, in order not to interfere with its antigen binding properties, thus narrowing candidate drugs to only the most potent classes of therapeutic agents.

Another advantage of the present invention derives from the fact that some metal-types target the most fundamental aspect of cancer cells, i.e., their rapidly dividing nature, whereas targeted approaches are contingent on interacting with specific features of particular cancer cells. Therefore, the efficacy of a nanocapsule comprising a bioactive therapeutic agent such as a nucleic acid, protein, peptide, or small molecule, can be enhanced and/or made clinically relevant when the nanocapsule comprises a metal treated polymer shell, as contemplated in one embodiment of the present invention.

The present invention demonstrates surprising synergy when the nanocapsule comprises both as metal-modified biocompatible polymer and a separate therapeutic cargo. When nanocapsule metal-modifications and therapeutic cargo were separately administered in mice at ultralow dosages which would not for the most part be expected to exert anti-proliferative effects, inhibition of tumor proliferation was relatively ineffective. In contrast, administration of ultralow dosages of the combination were highly effective. Thus, without wishing to be bound to any single theory, there appears to be more than an additive effect, (e.g., a synergistic effect) of the metal ion treatment of the biocompatible polymers of the present invention and the bioactive agent, on hyperproliferative cells.

The present invention is also surprising in view of the typical practice in the current art wherein metals are covalently bound to cell-targeting moieties. Covalent strategies are limited by a number of factors. As described above, one such limitation is the number of therapeutic agents that can be covalently linked to the carrier is relatively low, in order to not interefere with antigen binding properties of the carriers. Further, the linker systems must be both stable to prevent the drug from falling off in the blood, as well as cleavable to allow therapeutic activity upon reaching the interior of the cell. Moreover, linkages may render the complexes more prone to hydrolysis, complicating their use in the clinic. Additionally, because metals are electron deficient, conjugates with metals are typically susceptible to degradation by electron-rich protease, whereas in the present invention, without wishing to be bound to any single theory, it is believed the nanocapsules of the present invention are less susceptible to protease degradation because of the association between the metal composition and the biocompatible polymer. While some of the fundamental issues associated with covalent linking of carrier and metal can be and have been addressed, solutions add complexity and cost to the formulation and production processes.

In one embodiment, the present invention describes compositions and methods for effective and efficient delivery of macromolecules to target sites. In one aspect of the present invention, the targeting moiety is treated by a specific process involving metal ions which enhances the activity of the anti-proliferative bioactive agent (also known as the “cargo”). In certain embodiments of the invention, the compositions and methods are used to reduce metastatic burden. In certain embodiments of the invention, compositions and methods are used to deliver macromolecules to intracellular compartments via the caveolar pathway. This disclosure describes a nanoparticle vehicle comprising a metal ion-treated biocompatible polymer which optionally targets abnormally proliferative cells (hence, proliferative disease), including targeting of tumor cells. This disclosure also describes a novel therapeutic approach based upon the targeted delivery of pharmaceutical agents (e.g., small molecules or nucleic acids) to disseminated tumor cells using such a nanocapsule vehicle. Such methods can be used to effectively treat disseminated proliferative disease such as cancer. The nanocapsules are less than 50 nm in size, even when carrying, for example, a relatively large cargo (e.g., a 15 Kb plasmid).

Some of the methods for making particles useful for use with the instant invention have been previously disclosed in U.S. Pat. No. 6,632,771, U.S. Patent Application Publication No. 2004/0038303, U.S. Patent Application Publication No. 2007/0098713, U.S. Patent Application Publication No. 2004/0038406, U.S. Patent Application Publication No. 2004/0023855, PCT Publication No. WO06066154A2 and PCT Publication No. WO06065724A2 and PCT/US08/52863. Particles of the present invention are referred variously herein as “nanoparticles”, “particles”, “particles of the present invention”, “capsules”, “nanospheres”, and “nanocapsules”, or other such language, herein. In some instances, the term “capsules” or “nanocapsules” refers to moieties prior to the addition of the biocompatible polymer; context of use within the text will clarify whether the capsule or nanocapsule referred to refers to an entity prior to the addition of a biocompatible polymer.

In one embodiment, the present invention is directed to a method for forming particles useful for the treatment of proliferative disease, the method includes providing a bioactive component; providing a metal ion-treated biocompatible polymer component; coating the bioactive component with a surfactant having an HLB value of less than about 6.0 units under conditions which form a coated bioactive component; associating the coated bioactive component with the a metal ion-treated biocompatible polymer under conditions which associate the coated bioactive component with the metal-ion treated biocompatible polymer to form a particle. Particles created according to the instant method have an average diameter of less than about 50 nanometers as measured by atomic force microscopy of the particles following drying of the particles. In one embodiment, the particles have a mean surface charge of between about −15 and about +2 mev.

Specifically, as used herein, nanoparticles refer to stabilized surfactant micelles having an average diameter of between about 5 and 50 nanometers (i.e., “sub-50 nm nanocapsules”, or “s50 capsules”). An s50 capsule refers to a nanoparticle that has an approximate diameter of less than about 50 nm or an average diameter as discussed herein. In some embodiments, the particles (e.g., nanocapsules) have an average diameter of equal to or less than about 50 nm, equal to or less than about 45 nm, equal to or less than about 40 nm, equal to or less than about 35 nm, equal to or less than about 30 nm, equal to or less than about 25 nm, equal to or less than about 22 nm, equal to or less than about 20 nm, equal to or less than about 19 nm, equal to or less than about 18 nm, equal to or less than about 17 nm, equal to or less than about 16 nm, equal to or less than about 15 nm, equal to or less than about 14 nm, equal to or less than about 13 nm, equal to or less than about 12 nm, equal to or less than about 11 nm, equal to or less than about 10 nm, equal to or less than about 9 nm, equal to or less than about 8 nm, equal to or less than about 7 nm, equal to or less than about 6 nm, equal to or less than about 5 nm, or equal to or less than about 4 nm. In other embodiments, the particles have an average diameter of between about 5 nm and about 50 nm, between about 5 nm and about 45 nm, between about 5 nm and about 40 nm, between about 5 nm and about 35 nm, between about 5 nm and about 30 nm, between about 5 nm and about 25 nm, about between about 5 nm and about 20 nm, between about 5 nm and about 15 nm, between about 5 nm and about 12 nm, about between about 5 nm and about 11 nm, and between about 5 nm and about 9 nm. In other embodiments, the particles have an average diameter of between about 10 nm and about 50 nm, between about 10 nm and about 45 nm, between about 10 nm and about 40 nm, between about 10 nm and about 35 nm, between about 10 nm and about 30 nm, between about 10 nm and about 25 nm, about between about 10 nm and about 20 nm, and between about 10 nm and about 15 nm. In other embodiments, the particles have an average diameter of between about 15 nm and about 50 nm, between about 15 nm and about 45 nm, between about 15 nm and about 40 nm, between about 15 nm and about 35 nm, between about 15 nm and about 30 nm, between about 15 nm and about 25 nm, and between about 15 nm and about 20 nm.

Nanocapsules described herein can be targeted to tumors by coating the sub-50 nm nanocapsules with at least one tumor-specific targeting moiety. “Coating” includes “associating” a component (such as a bioactive agent, a surfactant, or a biocompatible polymer optionally capable of providing tumor specific targeting) with another component, and generally, but not always, refers to a non-covalent association and/or non-conjugation between components. In one embodiment, the bioactive component, surfactant, and metal ion treated-biocompatible polymer component are associated in a substantially non-covalent and/or non-conjugated association. In another embodiment, the bioactive component, surfactant, and metal ion treated-biocompatible polymer component are associated in a fully non-covalent and/or non-conjugated association.

Agents can include drugs, proteins, small molecules, toxins, hormones, enzymes, nucleic acids, peptides, steroids, growth factors, modulators of enzyme activity, modulators of receptor activity and vitamins.

In one embodiment, the method of the invention is practiced with a polynucleotide that inhibits a gene other than a Casein Kinase 2 (CK2) gene, including CK2 alpha (csnk2a1), CK2 alpha prime (csnk2a2), and/or CK2 beta (csnk2b), to treat prostate cancer and head neck cancer.

In another embodiment, the method of the invention is practiced with a polynucleotide that inhibits a gene other than a Casein Kinase 2 (CK2) gene, including CK2 alpha, CK2 alpha prime, and/or CK2 beta, to treat solid tumors. In another embodiment, the method of the invention is practiced with a polynucleotide that inhibits a gene other than a Casein Kinase 2 (CK2) gene, including CK2 alpha, CK2 alpha prime, and/or CK2 beta. In another embodiment, the method of the present invention is practiced with a nanocapsule that treats diseases other than cancer.

In another embodiment, the method of the present invention is practiced without a biocompatible polymer comprising tenascin-C or a fragment of tenascin-C. In another embodiment of the invention, the method of the present invention is practiced without a biocompatible polymer capable of targeting tenascin receptors or antigens.

In another embodiment, the method of the present invention is practiced with metal ion solutions that do not comprise the combination of arsenic, mercury, molybdenum, and selenium.

In one embodiment, a suitable method of making a particle is to form a dispersion of micelles by forming a coated bioactive agent (sometimes referred to herein as surfactant micelles), comprising a surfactant interfacing with a bioactive component, wherein the surfactant can have a hydrophile-lipophile-balance (HLB) value of less than about 6.0 units. Then the coated bioactive agent is dispersed into an aqueous composition, wherein the aqueous composition comprises a hydrophilic polymer (optionally, a metal ion-treated biocompatible polymer) so that the hydrophilic polymer associates with the coated bioactive agent to form particles. The particles may have an average diameter of less than about 50 nanometers, as described elsewhere herein.

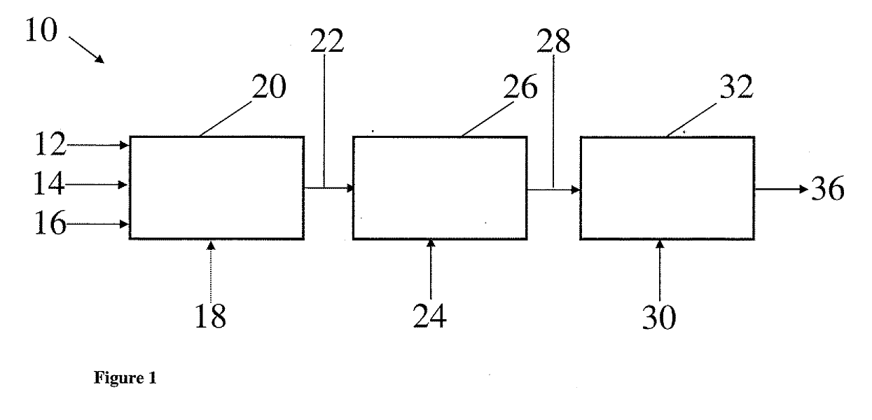

In general, any conventional apparatus and technique that is suitable for permitting the biocompatible polymer component 24 (FIG. 1) to stabilize the surfactant micelles 22 may be used as the stabilizing apparatus 26 in accordance with the present invention. Furthermore, any other device, such as high pressure homogenization or high ultrasound sonication is preferably not included during stabilization.

The biocompatible polymer component 24 may be supplied as individual biocompatible polymers or supplied in various prepared mixtures of two or more biocompatible polymers that are subsequently combined to form the biocompatible polymer component 24. A wide variety of polymers may be used as the biocompatible polymer, including many biologically compatible, water-soluble and water dispersible, cationic or anionic polymers. Due to an absence of water diffusion barriers, favorable initial biodistribution and multivalent site-binding properties, hydrophilic polymer components are typically useful for enhancing nanoparticle distribution in tissues. Any combination of any biocompatible polymer may be included in accordance with the present invention, while still realizing benefits of the present invention. Some non-exhaustive examples of biocompatible polymers include proteins, peptides, carbohydrates, glycoproteins, polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methylmethacrylate), poly(ethylmethacrylate), poly(butylmethacrylate), poly(isobutylmethacrylate), poly(hexlmethacrylate), poly(isodecylmethacrylate), poly(laurylmethacrylate), poly(phenylmethacrylate), poly(methylacrylate), poly(isopropylacrylate), poly(isobutacrylate), poly(octadecacrylate), polyethylene, polypropylene poly(ethylene glycol), poly(ethylene oxide), poly(ethylene terephthalate), poly(vinyl alcohols), poly(vinyl acetate, poly vinyl chloride, polystyrene, polyhyaluronic acids such as hyaluronan, casein, gelatin, gluten, polyanhydrides, polyacrylic acid, alginate, chitosan, any copolymerse thereof, and any combination of any of these.

Additionally, biocompatible polymers that have been modified for desirable enzymatic degradation, or change upon application of light, ultrasonic energy, radiation, a change in temperature, pH, osmolarity, solute or solvent concentration may also be included as part of the biocompatible polymer component 24. Preferably, the biocompatible polymer component 24 is a hydrophilic polymer that is capable of substantially coating, and preferably continuously coating the surfactant micelle 22. Still more preferably, the hydrophilic biocompatible polymer component 24 is capable of ionotophoretic exchange.

After associating the coated bioactive agent 22 with biocompatible polymer to form particles, the particles 28 may be transferred into a second aqueous composition 30 located in a second dispersing apparatus 32. The particles 28 may be transferred by mechanically forming droplets of the particles 28 that are subsequently introduced into the second aqueous composition 30.

The second aqueous composition 30 may include water only, or may optionally include a solute to precipitate the biocompatible polymer component 24 surrounding the stabilized surfactant micelle 28. Some non-exhaustive examples of solutes that may be used to precipitate the biocompatible polymer 24 include ionic species derived from elements listed in the periodic table.

Preferably, the second aqueous composition 30 includes a solute in an amount that is effective to solidify the biocompatible polymer component 24 and form the dispersed, and optionally atomized nanocapsules 36 of the present invention. As used herein, the term “solidify” refers to a solidifying or a hardening or a stabilizing of the biocompatible polymer component 24 that surrounds the stabilized surfactant micelles 28. It is also to be understood that the term “solidify” is also meant to encompass any crystallization of the biocompatible polymer 24 that may occur when the biocompatible polymer component 24 is exposed to the solute. Examples of cations for solidifying include, for example, Mn2+, Mg2+, Ca2+, A13+, Be2+, Li+, Ba2+, Gd3+, Sr3+. In one embodiment, to solidify the targeting moiety-adsorbed nanocapsule, the aqueous suspension of nanocapsules, such as, for example, nanocapsules comprising metal ion-coated targeting moieties, can be mixed into an aqueous solution of metal ions (i.e., a “stabilization solution”) capable of crystallizing or iontophoretic exchange with the coated nanocapsules.

The above-mentioned solidification step is separate from the step of metal-modification treatment of the biocompatible polymer prior to its association with (or incorporation into) the coated bioactive agent component. Representative and non-limiting examples of solutes that can be used to solidify the nanocapsules include ionic species derived from elements listed in the periodic table. Ions may be included in the aqueous stabilization composition in a range from 0.1 ppb to 1 Molar (M). An adequate amount of ion should be included such that the nanocapsules are sufficiently contacted with ions but not so much that aggregation occurs, which can lead to overly large capsules. In one embodiment, a solidification solution can include about 10 millimolar (mM) Ca2+ and about 200 mM Li+. If ultrapure reagents are used in the solidification solution, addition of very small amounts (e.g., less than 1 mM) of ions such as Ba, Fe, Mg, Sr, Pb and Zn, normally found in sufficient quantities in more standard preparations of lithium and calcium salts, may be added to optimize solidification of the coated nanocapsules. In one embodiment, a solidification solution includes 10 mM Ca2+, 200 mM Li+, and 1-500 nM of Sr+3 and Mg+2.

Optionally, after treatment with the solidification solution, nanocapsules have the morphology of a compact shape, which indicates optimized stability. An indication of optimized stability includes minimized surface area of the particles. Any configuration of the nanocapsule which gives rise to an overall compact and/or globular shape having an appropriate morphology, is acceptable, including where there is substructure to the nanocapsule, so long as the overall compact shape occurs. When the overall morphology of the nanocapsule comprises a compact shape, the final surface charge approaches about neutral or slightly negative charge. Final surface charge of neutral or slightly negative is a preferred surface charge for the particles of the present invention. More specifically, we have found that particles with charge of between about +2 and ±15 milli electron volts (mev) are desirable. Additionally, any other components that are capable of increasing the stability of the nanocapsules can be included as part of the stabilization solution such that the final dry average diameter of the nanocapsules is between a range of 5-50 nm by AFM.

The particles 28 may be transferred into the second aqueous composition 30 via atomization through a nozzle (not shown) having a particular orifice size or through an aerosolizing apparatus (not shown). Atomizing or aerosolizing the particles 28 typically includes the application of a shear force that may be capable of further dispersing the particles 28. Furthermore, the application of the shear force during transfer may also be effective to (1) reduce the size of the nanocapsules 36, or (2) break up any agglomerates or associations between particles 28 that may have formed in the stabilizing apparatus 26. Feed pressures of less than about 100 psi, for example, may be used to atomize the particles 28.

After stabilizing and/or optionally incubating the nanocapsules 36 in the second aqueous composition 30, the nanocapsules 36 may be filtered, centrifuged or dried to obtain separate and discrete nanocapsules 36. In one embodiment, nanocapsules are atomized through a nozzle, to increase uniformity of size. Atomization should be sufficient to apply a shear force capable of breaking up flocculated aggregates without so much force as to induce hard aggregates. Those skilled in the art will understand that a particular nozzle diameter will lead to range of feed pressures suitable for atomizing the nanocapsules to a suitable and consistent size. In one embodiment, a nozzle diameter of less than about 250 microns with feed pressures of less than about 10 psi produces suitable nanocapsules. In some embodiments, the nanocapsules can be atomized into a stabilization solution.

The incubation time and temperature may be varied from about 8 hr to 7 days to vary the amount of time required for particle dissolution or disassembly in end use. After precipitating, atomizing, and/or incubating the nanocapsules in a stabilization solution, the nanocapsules can be filtered, centrifuged and/or dried to obtain separate and discrete sub-50 nm nanocapsules. In one embodiment, nanocapsules are incubated for 2 days at about 4° C. The resultant nanocapsules can be frozen or dried and reconstituted for later use.

The following advantages are inherent in nanocapsules. Having a diameter of less than about 50 nm enhances delivery of bioactive components by protecting the bioactive components against degradation during transport to the target cell. Uptake of the nanocapsules 36 by the target cell occurs via transport systems, such as a non-endosomal pathway, that prevents lysosomal degradation of the nanocapsules 36. As discussed above, nanocapsules are efficiently exported into a cell via a caveolin-regulated pathway that circumvents most, if not all, endosomal-regulated pathways that typically degrade nanocapsules 36. In addition, the neutral or net negative charge of the nanocapsules promotes long serum half-life and prevents the negative effects associated with, for example, positively-charged non-viral delivery vehicles such as accumulation of serum proteins via charge interactions. Accumulation of serum proteins can increase the apparent size of the vehicle and may thereby alter the tissue specificity and uptake, and is therefore undesirable.

The nanocapsules 36 may be combined with additional polymeric binders, surfactants, fillers, and other excipients to incorporate the nanocapsules 36 into solid dosage forms such as granules, tablets, pellets, films or coatings for use in enhanced bioactive component 12 delivery. In this way, design of the dissolution profile, control of the particle size, and cellular uptake remains at the level of the nanocapsule. Such applications include, but are not limited to, creation of rapidly dissolving pellets of nanocapsules for pulmonary delivery or nanocapsule films for device-mediated delivery.

Nanoparticles are described herein that are configured to enter cells via caveolae, a mechanism for cell entry that has many advantages compared to other entry mechanisms. Moreover, such nanoparticles are so small that they penetrate the spaces between cells and move freely through tissues. Indeed, nanoparticles of less than about 70 or 50 nm in diameter are much smaller than the spaces between cells. For example, suitably sized nanoparticles may pass out of blood vessels through the spaces between endothelial cells that line the blood vessels, and into the vascular media. Thus intravascular delivery of suitably sized nanoparticles allows for the nanoparticles to be delivered to tissues beyond the vasculature.

In one specific embodiment, the aqueous solution of the bioactive agent (e.g., a cargo moiety, either complexed or uncomplexed as described elsewhere herein) can be associated with a surfactant (e.g., encapsulated) by first dispersing the cargo moiety into a biocompatible, water-miscible solvent using a biocompatible, water-insoluble surfactant system suitable for preparation of an inverted or reverse micelle. As discussed elsewhere herein, suitable surfactant systems are well-known in the formulation arts as amphillic materials that are essentially hydrophobic and characterized by a hydrophile-lipophile balance (HLB) of less than about 6, a critical micelle concentration (CMC) of less than about 200 μM, and/or a critical packing diameter greater than 1. Hydrophobic surfactants and hydrophobic, water-miscible solvents suitable for preparing reverse micelles are described in Pashley & Karaman (2004, In Applied Colloid and Surface Chemistry, John Wiley, pgs 60-85), Rosen (2004, In Surfactants and Interfacial Phenomena, John Wiley), The Handbook of Industrial Surfactants (1993, Ash, ed., Gower Pub), and Perry's Chemical Engineer's Handbook (1997, Perry & Green, 7th Ed., McGraw-Hill Professional). In one embodiment, a hydrophobic surfactant can be 2,4,7,9-tetramethyl-5-decyn-4,7-diol (TM-diol) used in a concentration of up to 0.5% by weight of surfactant micelle volume, and a water-miscible solvent can be DMSO. The concentration of surfactant selected should be sufficient to prepare an optically clear nanoemulsion but not so much as to induce aggregation, since aggregation can lead to overly large nanocapsules. In one embodiment, the includes at least one of cetyl alcohol, 2, 4, 7, 9-tetramethyl-5-decyn-4,7-diol, molecules containing an acetylenic diol portion, and blends of 2, 4, 7, 9-tetramethyl-5-decyn-4,7-diol.

In this embodiment, the coated bioactive agent (also referred to herein as micelles carrying the cargo moieties, and also referred to herein as nanoparticles) can be associated with and/or coated with tumor-targeting moieties (e.g., tenascin polypeptides) by mixing one or more targeting moieties with an aqueous dilution of the nanocapsules. In one embodiment the targeting moieties can be mixed with nanocapsules in a ratio (by weight) of about 1:100 to about 1:0.1 of nanocapsule to targeting moiety, depending upon the rate at which the nanocapsule is desired to dissolve or disassemble. In one embodiment, the coating weight ratio is 1:16 of nanocapsules to targeting moieties. Nanoparticles can comprise various targeting components, e.g., ligands, to target the nanoparticle and its contents to, e.g., specific cells. The contents of the nanoparticle may be, for example, therapeutic agents that alter the activity of the cell, or a marker. The biocompatible polymer can comprise the ligand, or the ligand can be otherwise incorporated into the nanoparticles. For example, if one more than one type of cell is being cultured, a particular cell type or subset of cells may be targeted using nanoparticles having ligands that are specific to particular targets on the cells. Thus, for example, several cells in the field of view of a microscope may be observed while a subset of the cells are undergoing treatment. Thus some of the cells serve as controls for the treated cells. Or, cells may advantageously be treated while cultured with other cells, for example, some cultured stem cells are known to be advantageously grown in co-culture with other cell types. Table A and Table B set forth some non limiting examples of targeting components for particles of the invention, and examples of cell recognition components specific for cell recognition targets. A ligand or targeting component or cell recognition component is a molecule that specifically binds to another molecule, which may be referred to as a target or a cell recognition target. Thus a ligand for a growth factor receptor may be, e.g., a growth factor, a fragment of a growth factor, or an antibody. Those of ordinary skill in these arts are able to distinguish specific binding from non-specific binding; for example, the identification of a ligand for a cell receptor requires distinguishing it from other molecules that nonspecifically bind the receptor.

Targeting components and/or agents delivered using nanoparticles may be copolymerized, linked to, fused with, or otherwise joined or associated with other molecules, e.g., see Halin et. al, Nature Biotech. (2002) 20:264-69, “Enhancement of the antitumor activity of interleukin-12 by targeted delivery to neovasculature” for a review of fusion proteins. Moreover, antibodies (described below) or peptides may be developed to target specific tissues. For example, a screening assay may be performed using a library and a target. Thus a library of potential ligands may be screened against targets, e.g., tumor tissue. An example of a screening method is set forth in U.S. Pat. No. 6,232,287, which describes various phage panning methods, both in vitro and in vivo. Such peptides may be incorporated into nanoparticles for targeting uses.

TABLE A
Targeting components for particles

| Target cell | Targeting component | Reference/Source |
|---|---|---|
| Endothelial cells | Albumin | U.S. Pat. No. 6,204,054. (for trancytosis) |
| Keratinocytes | Laminin | Glia 8: 71 |
| Tumor cells | thrombospondin (TSP) | Wang et. al, Am. J Surg. 170(5) 502-5 |
| | Osteopontin (OP) | Senger et. al, Ann NY Acad Sci 760: 83-100 |
| | Thrombin-cleaved OP | |
| | Fibronectin | Unger et. al, 2001, AAPS Pharmsci 3(3) |
| | | Supplement: 3731 |
| Myocytes | Fibronectin, Laminin | Hornberger, Circ Res. 87(6): 508-15 |
| | β1d integrin ligands | Am. J. Phys. 279(6): H2916-26 |
| | PVP 10,000 MW | |
| hepatocytes/liver | DGEA peptide | Sponsel et. al, Am J. Phys 271: c721-c272 |
| cells | | |
| hepatic stellate | Collagen, laminin | Gastroent 110: 1127-1136 |
| chondrocytes/bone | Osteopontin | Cell Ad Commun 3: 367-374, U.S. Pat. No. 6,074,609, |
| cells | | U.S. Pat. No. 5,770,565, PCT W0 0980837A1, PCT |
| | | W0 0209735A2 |
| | BMP | U.S. Pat. No. 6,352,972 |
| | SPARC/osteonectin | PCT W0072679a1 |
| | collagen2 | PCT W0 145764a1 |
| | HA | U.S. Pat. Nos. 51,283,26 & 5,866,165 |
| | Osteocalcin | U.S. Pat. No. 6,159,467 |
| Smooth muscle cells | Osteopontin | U.S. Pat. No. 5,849,865 |
| Stem cells | FN, rE-selectin, HA | Kronenwett et. al, Stem Cells 18(5)320-330 |
| Neurons | Nerve Growth Factor, | Development 124(19): 3909-3917 |
| | Agrin | |
| | contactin ligand | U.S. Pat. No. 5,766,922 |
| | NCAM, L1 | U.S. Pat. No. 5,792,743 |
| | KAL | U.S. Pat. No. 6,121,231 |
| | Phosphacan | U.S. Pat. No. 5,625,040 |
| | Neurocan | U.S. Pat. No. 5,648,465 |
| | Cytotactin | U.S. Pat. No. S 6,482,410 |
| | Laminin, KS- and β1k | U.S. Pat. No. 5,610,031 |
| | chain | U.S. Pat. No. 5,580,960 |
| | Merosin | U.S. Pat. No. 5,872,231 |
| Schwann | Ninjurin | U.S. Pat. No. 6,140,117 |
| cells/neuron | | |
| Retinal ganglion | Osteonectin | J. Histochem Chem 46(1): 3-10 |
| | Laminin | Dev. Biol. 138: 82-93 |
| Muller cells | rNcam, r L-1 rN-cadherin | Dev. Biol. 138(1): 82-93 |
| Blood-Brain barrier | Peptide vectors e.g. d- | Rouselle et. al, Molecular Pharmacology, |
| | penetratin, pegelin, | (2000) 57: 679-686 |
| | protegrins and related | |

TABLE A2
Additional Candidate Excipients for angiogenic and
anti-tumor particle targeting agents

| | Potential Role in Tumor | |
|---|---|---|
| Candidate Particle Material | Biology | Reference |
| Recombinant Pex binding | Extravasation of tumor cells | Bello et. al, Cancer Research |
| domain of membrane- | from bloodstream into distant | (2001) 61: 8730-36 |
| associated Matrix | site from primary tumor | |
| Metalloproteinase-1 | | |
| Bovine bone-derived | Chemokine attracting | Jacob et. al, Cancer Research |
| Osteonectin | metastatic tumor cells to bone | (1999) 59: 4453-57 |
| Fibronectin inhibitory | Blocks α₅β₁ integrin binding | Livant et. al, Cancer |
| peptide, PHSCN | site on migrating tumor cells, | Research (2000) 60: 309- |
| | preventing tissue | |
| | extravasation | |
| Recombinant truncated | Modified ligand for CEA | PCT W0 02100343A2 |
| Galectin-3 | antigen, plays role in tumor | Glinsky et. al, Cancer |
| | cell extravasation | Research (2001) 61: 4851-57 |
| Hyaluronan | Feature of tumor stroma, | Simpson et. al, J Biol. Chem |
| | plays role in tumor | (2001) 276(21): 17949-57 |
| | extravasation | |
| Tenascin | Feature of tumor stroma | Tuxhorn et. al, J Urol. (2001) |
| | | 166: 2472-2483 |

Embodiments include, for example, nanoparticles and particles that comprise ligands that bind to cellular adhesion molecules and thereby target the nanoparticle and its contents to specific cells. Various cell surface adhesion molecules are active in numerous cellular processes that include cell growth, differentiation, development, cell movement, cell adhesion, and cancer metastasis. There are at least four major families of cell adhesion molecules: the immunoglobulin (Ig) superfamily, integrins, cadherins, and selecting. Cell adhesion molecules are critical to numerous cellular processes and responses. Additionally, they also play a role in various disease states. For example, tumorigenesis is a process that involves cell adhesion molecules. For successful tumorigenesis, there must be changes in cellular adhesivity which facilitate the disruption of normal tissue structures. Cell adhesion molecules are objects of intense study and improved tools for use with these molecules are required for in vitro and in vivo applications.

Members of the Ig superfamily include the intercellular adhesion molecules (ICAMs), vascular-cell adhesion molecule (VCAM-1), platelet-endothelial-cell adhesion molecule (PECAM-1), and neural-cell adhesion molecule (NCAM). Each Ig superfamily cell adhesion molecule has an extracellular domain, which has several Ig-like intrachain disulfide-bonded loops with conserved cysteine residues, a transmembrane domain, and an intracellular domain that interacts with the cytoskeleton. The Ig superfamily cell adhesion molecules are calcium-independent transmembrane glycoproteins.

Integrins are transmembrane proteins that are constitutively expressed but require activation in order to bind their ligand. Many protein and oligopeptide ligands for integrins are known. Integrins are non-covalently linked heterodimers having alpha and beta subunits. About 15 alpha subunits and 8 beta subunits have been identified. These combine promiscuously to form various types of integrin receptors but some combinations are not available, so that there are subfamilies of integrins that are made of various alpha and beta combinations. Integrins appear to have three activation states: basal avidity, low avidity, and high avidity. Additionally, cells will alter integrin receptor expression depending on activation state, maturity, or lineage.

The cadherins are calcium-dependent adhesion molecules and include neural (N)-cadherin, placental (P)-cadherin, and epithelial (E)-cadherin. All three belong to the classical cadherin subfamily. There are also desmosomal cadherins and proto-cadherins. Cadherins are intimately involved in embryonic development and tissue organization. They exhibit predominantly homophilic adhesion, and the key peptidic motifs for binding have been identified for most cadherins. The extracellular domain consists of several cadherin repeats, each is capable of binding a calcium ion. Following the transmembrane domain, the intracellular domain is highly conserved. When calcium is bound, the extracellular domain has a rigid, rod-like structure. The intracellular domain is capable of binding the a, b, and g catenins. The adhesive properties of the cadherins have been shown to be dependent upon the ability of the intracellular domain to interact with cytoplasmic proteins such as the catenins.

The selectins are a family of divalent cation dependent glycoproteins that bind carbohydrates, binding fucosylated carbohydrates, especially, sialylated Lewisx, and mucins. The three family members include: Endothelial (E)-selectin, leukocyte (L)-selectin, and platelet (P)-selectin. The extracellular domain of each has a carbohydrate recognition motif, an epidermal growth factor (EGF)-like motif, and varying numbers of a short repeated domain related to complement-regulatory proteins (CRP). Each has a short cytoplasmic domain. The selectins play an important role in aspects of cell adhesion, movement, and migration.

TABLE B
Examples of Cell Recognition Components Specific for Cell Recognition Targets

| | Alternative | | |
|---|---|---|---|
| Targeting | Names | | Example of Tumor |
| Ligands | (trade name) | Target | Target |
| RGD peptide | | Cellular adhesion | Vasculature endothelial |
| | | molecules, such as ανβ3- | cells in solid tumors |
| | | integrin | |
| NGR | | Aminopeptidase N | Vasculature endothelial |
| | | (CD13) | cells in solid tumors |
| Folate | | Folate receptor | Cancer cells that |
| | | | overexpress the folate |
| | | | receptor |
| Transferrin | | Transferrin receptor | Cancer cells that |
| | | | overexpress the |
| | | | transferrin receptor |
| GM-CSF | | GM-CSF receptor | Leukaemic blasts |
| Galactosamine | | Galactosamine receptors | Hepatoma |
| | | on hepatocytes | |
| Anti-VEGFR | 2C3 | Vasculature endothelial | Vasculature endothelial |
| antibody | | growth-factor receptor | cells in solid tumors |
| | | (FLK1) | |
| Anti-ERBB2 | Trastuzumab | ERBB2 receptor | Cells that overexpress |
| antibody | (Herceptin) | | the ERBB2 receptor, |
| | | | such as in breast and |
| | | | ovarian cancers. |
| Anti-CD20 | Rituximab | CD20, a B-cell surface | Non-Hodgkin's |
| antibody | (Rituxan), | antigen | lymphoma and other B- |
| | ibritumomab | | cell lymphoproliferative |
| | tiuxetan (Zevalin) | | diseases |
| Anti-CD22 | Epratuzumab, | CD22, a B-cell surface | Non-Hodgkin's |
| antibody | LL2, RFB4 | antigen | lymphoma and other B- |
| | | | cell lymphoproliferative |
| | | | diseases |
| Anti-CD19 | B4, HD37 | CD19, a pan-B-cell | Non-Hodgkin's |
| antibody | | surface epitope | lymphoma and other B- |
| | | | cell lymphoproliferative |
| | | | diseases |
| Anti-CD33 | Gemtuzumab, | CD33, a sialo-adhesion | Acute myeloid leukemia |
| antibody | ozogamicin | molecule, leukocyte | |
| | (Mylotarg) | differentiation antigen | |
| Anti-CD33 | M195 | CD33, a T-cell epitope | Acute myeloid leukemia |
| Anti-CD25 | Anti-Tac, LMB2 | CD25, α-subunit of the | Hairy-cell leukaemia, |
| | | interleukin-2 receptor on | Hodgkin's and other |
| | | activated T cells | CD25⁺ lymphoma |
| | | | haematological |
| | | | malignancies |
| Anti-CD25 | Denileukin | Interleukin-2 receptor | Cutaneous T-cell |
| | diftitox (Ontak) | | lymphoma |
| Anti-HLA- | Lym1 | HLA-DR10β subunit | Non-Hodgkin's |
| DR10β | | | lymphoma and other B- |
| | | | cell lymphoproliferative |
| | | | diseases |
| Anti-tenascin | 81C6 | Extracellular-matrix | Glial tumors, breast |
| | | protein overexpressed in | cancer |
| | | many tumors | |
| Anti-CEA | MN-14, F6, | CEA | Colorectal, small-cell |
| | A5B7 | | lung and ovarian cancers |
| Anti-MUC1 | HMFG1, BrE3 | MUC1, an aberrantly | Breast and bladder |
| | | glycosylated epithelial | cancer |
| | | mucin | |
| Anti-TAG72 | CC49, B72.3 | TAG72, oncofetal antigen | Colorectal, ovarian and |
| | | tumor-associated | breast cancer |
| | | glycoprotein-72 | |

Embodiments include, for example, nanoparticles associated with growth factors so that the nanoparticles are specifically targeted to cells expressing the growth factor receptors. Other embodiments include nanoparticles having growth factors that are delivered to the cell to modulate the activity of the cell. Other embodiments include ligands that specifically bind to growth factor receptors so as to specifically target the nanoparticle to cells having the growth factor receptor. The following list of polypeptides include polypeptides that are suitable biocompatible polymer and suitable cell recognition polypeptides.

Growth factors are suitable cell recognition polypeptides according to the invention. Growth factors are active in many aspects of cellular and tissue regulation including proliferation, hyperproliferation, differentiation, trophism, scarring, and healing, as shown in, for example, Table 3. Growth factors specifically bind to cell surface receptors. Many growth factors are quite versatile, stimulating cellular activities in numerous different cell types; while others are specific to a particular cell-type. Targeting nanoparticles to a growth factor receptor enables the activity of the cell to be controlled. Thus many aspects of physiological activity may be controlled or studied, including proliferation, hyperproliferation, and healing. A growth factor refers to a growth factor or molecules comprising an active fragment thereof, and includes purified native polypeptides and recombinant polypeptides.

Nanoparticles may be targeted to growth factor receptors by a variety of means. For example, antibodies against the receptor may be created and used on the nanoparticles for direction specifically to the receptor. Or, the growth factor, or a fragment thereof, may be used on the nanoparticles to directed specifically to the receptor. The blinding of growth factors to growth factor receptors has, in general, been extensively studied, and short polypeptide sequences that are a fragment of the growth factors, and bind to the receptors, are known.

For example, if it is desirable to limit the proliferation of glial or smooth muscle cells, a particle associated with a cell behavior modulating agent, e.g., a toxin or antiproliferative agent, may be decorated with a ligand that specifically binds PDGF-R (Table C). Table C provides non limiting examples of growth factors and growth factor receptors for cell and tissue targeting. Since PDGF-R is preferentially expressed by glial or smooth muscle cells, the particles will preferentially be taken up by glial or smooth muscle cells. The toxin would kill the cells or the antiproliferative agent would reduce proliferation. Similarly, other cellular activities, e.g., as set forth in Table C, may be controlled by specifically targeting nanoparticles having modulating agents.

TABLE C
Growth Factors and Growth Factor Receptors for Cell and Tissue Targeting

| Factor | Receptor | Source | Activity | Comments |
|---|---|---|---|---|
| PDGF | PDGF-R | platelets, | proliferation of | two different |
| | | endothelial | connective | protein chains |
| | | cells, placenta | tissue, glial and | form 3 distinct |
| | | | smooth muscle | dimer forms; AA, |
| | | | cells | AB and BB |
| EGF | EGF-R | submaxillary | proliferation of | |
| | | gland, Brunners | mesenchymal, | |
| | | gland | glial and | |
| | | | epithelial cells | |
| TGF-a | TGF-a-R | common in | active for normal | related to EGF |
| | | transformed | wound healing | |
| | | cells | | |
| FGF | FGF-R | wide range of | promotes | at least 19 family |
| | | cells; protein is | proliferation of | members, 4 |
| | | associated with | many cells; | distinct receptors |
| | | the ECM | inhibits some | |
| | | | stem cells | |
| NGF | NGF-R | | promotes neurite | related proteins |
| | | | outgrowth and | identified as |
| | | | neural cell | proto-oncogenes; |
| | | | survival | trkA, trkB, trkC |
| Erythropoietin | Erythropoietin-R | kidney | promotes | |
| | | | proliferation and | |
| | | | differentiation of | |
| | | | erythrocytes | |
| TGF-b | TGF-b-R | activated TH₁ | anti- | at least 100 |
| | | cells (T-helper) | inflammatory, | different family |
| | | and natural | promotes wound | members |
| | | killer (NK) cells | healing, inhibits | |
| | | | macrophage and | |
| | | | lymphocyte | |
| | | | proliferation | |
| IGF-I | IGF-I-R | primarily liver | promotes | related to IGF-II |
| | | | proliferation of | and proinsulin, |
| | | | many cell types | also called |
| | | | | Somatomedin C |
| IGF-II | IGF-II-R | variety of cells | promotes | related to IGF-I |
| | | | proliferation of | and proinsulin |
| | | | many cell types | |
| | | | primarily of fetal | |
| | | | origin | |

Epidermal growth factor (EGF), like all growth factors, binds to specific high-affinity, low-capacity cell surface receptors. Intrinsic to the EGF receptor is tyrosine kinase activity, which is activated in response to EGF binding. EGF has a tyrosine kinase domain that phosphorylates the EGF receptor itself (autophosphorylation) as well as other proteins, in signal transduction cascades. Experimental evidence has shown that the Neu proto-oncogene is a homologue of the EGF receptor, indicating that EGF is active in cellular hyperproliferation. EGF has proliferative effects on cells of both mesodermal and ectodermal origin, particularly keratinocytes and fibroblasts. EGF exhibits negative growth effects on certain carcinomas as well as hair follicle cells. Growth-related responses to EGF include the induction of nuclear proto-oncogene expression, such as Fos, Jun and Myc.

Fibroblast Growth Factors (FGFs) are a family of at least 19 distinct members. Kaposi's sarcoma cells (prevalent in patients with AIDS) secrete a homologue of FGF called the K-FGF proto-oncogene. In mice the mammary tumor virus integrates at two predominant sites in the mouse genome identified as Int-1 and Int-2. The protein encoded by the Int-2 locus is a homologue of the FGF family of growth factors. A prominent role for FGFs is in the development of the skeletal system and nervous system in mammals. FGFs also are neurotrophic for cells of both the peripheral and central nervous system. Additionally, several members of the FGF family are potent inducers of mesodermal differentiation in early embryos. The FGFs interact with specific cell-surface receptors that have been identified as having intrinsic tyrosine kinase activity. The Flg proto-oncogene is a homologue of the FGF receptor family. FGFR3 is predominantly expressed in quiescent chondrocytes where it is responsible for restricting chondrocyte proliferation and differentiation. In mice with inactivating mutations in FGFR3 there is an expansion of long bone growth and zones of proliferating cartilage further demonstrating that FGFR3 is necessary to control the rate and amount of chondrocyte growth.

Platelet-Derived Growth Factor (PDGF) has two distinct polypeptide chains, A and B. The c-S is proto-oncogene has been shown to be homologous to the PDGF A chain. Like the EGF receptor, the PDGF receptors have autophosphorylating tyrosine kinase activity. Proliferative responses to PDGF action are exerted on many mesenchymal cell types. Other growth-related responses to PDGF include cytoskeletal rearrangement and increased polyphosphoinositol turnover. PDGF induces the expression of a number of nuclear localized proto-oncogenes, such as Fos, Myc and Jun.

Transforming Growth Factors-β (TGFs-β) was originally characterized as a protein (secreted from a tumor cell line) that was capable of inducing a transformed phenotype in non-neoplastic cells in culture, and thus is implicated in numerous hyperproliferation disorders. The TGF-β-related family of proteins includes the activin and inhibin proteins. The Mullerian inhibiting substance (MIS) is also a TGF-β-related protein, as are members of the bone morphogenetic protein (BMP) family of bone growth-regulatory factors. Indeed, the TGF-β family may comprise as many as 100 distinct proteins, all with at least one region of amino-acid sequence homology. There are several classes of cell-surface receptors that bind different TGFs β with differing affinities. The TGF-β family of receptors all has intrinsic serine/threonine kinase activity and, therefore, induce distinct cascades of signal transduction. TGFs-βs have proliferative effects on many mesenchymal and epithelial cell types and sometimes demonstrate anti-proliferative effects on endothelial cells.

Transforming Growth Factor-α (TGF-α was first identified as a substance secreted from certain tumor cells that, in conjunction with TGF-β1, could reversibly transform certain types of normal cells in culture, and thus is implicated in numerous hyperproliferative disorders. TGFα. binds to the EGF receptor, as well as its own distinct receptor, and it is this interaction that is thought to be responsible for the growth factor's effect. The predominant sources of TGF-α are carcinomas, but activated macrophages and keratinocytes (and possibly other epithelial cells) also secrete TGFα. In normal cell populations, TGFα is a potent keratinocyte growth factor.

Tumor Necrosis Factor-β (TNF β)s are suitable cell recognition polypeptides according to the invention. TNFβ (also called lymphotoxin) is characterized by its ability to kill a number of different cell types, as well as the ability to induce terminal differentiation in others. One significant non-proliferative response to TNF-β is an inhibition of lipoprotein lipase present on the surface of vascular endothelial cells. The predominant site of TNF-β synthesis is T-lymphocytes, in particular the special class of T-cells called cytotoxic T-lymphocytes (CTL cells). The induction of TNF-β expression results from elevations in IL-2 as well as the interaction of antigen with T-cell receptors.

Embodiments can be particles, e.g., nanoparticles, associated with extracellular matrix molecules so that the particles are specifically targeted to cells expressing receptors for the extracellular matrix molecules. Alternatively, particles may comprise ligands for the extracellular matrix molecules so that the particles become associated with the extracellular matrix molecules on tissues or cells. Extracellular matrix molecules are suitable cell recognition polypeptides according to the invention. The extracellular matrix comprises a variety of proteins and polysaccharides that are assembled into organized matrices that form the scaffold of tissues. The common components of the extracellular matrix can be referred to as extracellular matrix molecules. Examples of extracellular matrix molecules are tenacin, collagen, laminin, fibronectin, hyaluronic acid, chondroitin sulfate, dermatan sulfate, heparin sulfate, heparin, keratan sulfate, elastin, vitronectin, and subtypes thereof. Cells typically secrete extracellular matrix molecules in response to their environments, so that the patterns of extracellular matrix molecule expression may be indicative of certain conditions. For example, EDA, a domain of fibronectin may be targeted for cancer.

Aberration in the patterns of expression of extracellular matrix molecules can indicate pathological conditions. For example, human tenascin is an extracellular matrix molecule, a 240.7 kDa glycoprotein. Tenascin is found in abundance in embryonic tissue, whereas the expression in normal adult tissue is limited. Tenascin has been reported to be expressed in the stroma of many tumors, including gliomas, breast, squamous cell and lung carcinomas. Thus it is possible to control hyperproliferative conditions, including many tumors, by specifically directing therapeutic agents to tenascin.

Embodiments can be particles, e.g., nanoparticles, associated with extracellular matrix molecules so that the particles are specifically targeted to cells expressing receptors for the extracellular matrix molecules. Nanocapsules targeted to the extracellular matrix are useful for variety of therapeutic, scientific, and research applications. For example, extracellular matrix molecules specifically bind to receptors on cells, so that nanoparticles comprising extracellular matrix molecules are thereby targeted to extracellular matrix molecule receptors. Further, drugs may be targeted to the extracellular matrix by making nanoparticles having ligands and/or coatings that bind extracellular matrix molecules. Moreover, particles having a visualization agents directed to extracellular matrix molecules may be used for microscopy, e.g. fluorescence or histochemistry.

Small molecules and/or toxins are a type of agent that may be loaded into a nanoparticle and delivered to a cell or tissue. Many small molecules are known to those of skill, including those for use in treating cancer. Embodiments include nanoparticles loaded with small molecule toxins. Suitable small molecule toxins include, for example: alkylators such as asaley, AZQ, BCNU, busulfan, carboxyphthalatoplatinum, CBDCA, CCNU, CHIP, chlorambucil, chlorozotocin, clomesone, cyclodisone, cyclophosphamide, dacarbazine, dianhydrogalactitol, fluorodopan, hepsulfam, hycanthone, L-PAM, melphalan, methyl CCNU, mitomycin C, mitozolamide, nitrogen mustard, PCNU, piperazine alkylator, piperazinedione, pipobroman, porfiromycin, spirohydantoin mustard, temozolomide, teroxirone, tetraplatin, thio-tepa, triethylenemelamine, uracil nitrogen mustard, and Yoshi-864; anthracyclines such as doxorubicin, cyanomorpholinodoxorubicin, mitoxantrone, idarubicin, valrubicin, epirubicin, daunomycin, antibiotics such as dactinomycin, actinomycin D, bleomycin, and daunorubicin; aromatase inhibitors such as anastrozole and letrozole; covalent conjugate of recombinant methionyl human GCSF and monomethoxypolyethylene glycol; cyclo-oxygenase inhibitors such as celecoxib; estrogen receptor modulators such as tamoxifen and fulvestrant; folate antagonists such as methotrexate; hormonals such as anastrozole; inorganic arsenates such as arsenic trioxide; microtubule inhibitors such as vincristine, vinblastine, paclitaxel, vinorelbine, and docetaxel; modifiers such as leucovorin and dexrazoxane; nitrosoureas such as procarbazine, lomustine, CCNU, carmustine, estramustine; nucleoside analogues such as mercaptopurine, 6-MP, fluorouracil, 5-FU, thioguanine, 6-TG, cytarabine, floxuridine (intraarterial), fludarabine, pentostatin, cladribine, pentostatin, capecitabine, gemcitabine, and cytarabine; oxaliplatin; retinoids such as tretinoin, ATRA, alitretinoin, and bexarotene capsules gel; stem cell stimulators such as Oprelvekin; topoisomerase 1 inhibitors such as topotecan and irinotecan; topoisomerase 2 inhibitors such as etoposide, (VP-16), teniposide, (VM-26), and etoposide phosphate; tyrosine kinase inhibitors such as imatinib mesylate; urate-oxidase enzymes such as Rasburicase; and hydroxyurea. In one embodiment, suitable toxins include 2-dimethylamino-4,5,6,7-tetrabromo-1H-benzimidazole (DMAT), cisplatin, anthracyclines, doxorubicin, vincristine, cyclophosphamide, topotecan, taxol, and paclitaxel. These small toxins are, in general, predominantly hydrophobic and have relatively low MWs, about 1000 or less. Moreover, peptidic oncoagents are contemplated. Further, compounds and agents that have been shown to be useful for modulating cellular activities for a therapeutic or diagnostic use are contemplated. For example, PCT WO 02/100343 describes the use of galectin for hyperproliferative disorders.

Embodiments include nanoparticles and particles that comprise agents that modulate apoptosis, for example, by reducing or increasing the incidence of apoptosis. Apoptosis plays a major role during development and homeostasis. Apoptosis can be triggered in a variety of cell types by the deprivation of growth factors, which appear to repress an active suicide response. Inducing apoptosis in cancer cells can be an effective therapeutic approach. Inducing apoptosis in tissue cultured cells provides a model system for studying the effects of certain drugs for triggering, reversing, or halting the apoptotic pathway. Accordingly, increasing a cell's potential to enter the apoptotic pathway, or otherwise modulating apoptosis, is useful. It is contemplated that the ability to inhibit apoptosis in a eukaryotic cell in tissue culture provides a model system for testing certain proteins and factors for their role in the apoptotic pathway. It also provides a model system for testing compounds suspected of being tumorigenic. In vitro such oligonucleotide containing nanoparticles may be administered by topical, injection, infusion or static coculture. In vivo administration of oligonucleotide containing nanoparticles can be subdermal, transdermal, subcutaneous, or intramuscular. Intravenous administration or use of implanted pumps may also be used. Doses are selected to provide effective inhibition of cancer cell growth and/or proliferation.

Specifically, some factors for modulating apoptosis include factors that activate or deactivate death receptors, including ligands for death receptors or factors that competitively inhibit the finding of factors to death receptors. Thus there are many factors that are modulators of apoptosis, i.e., that serve to enhance, inhibit, trigger, initiate, or otherwise affect apoptosis. Apoptosis may be triggered by administration of apoptotic factors, including synthetic and natural factors. Some natural factors interact with cell surface receptors referred to death receptors and contribute to, or cause, apoptosis. Death receptors belong to the tumor necrosis factor (TNF) gene superfamily and generally can have several functions other than initiating apoptosis. The best characterized of the death receptors are CD95 (or Fas), TNFR1 (TNF receptor-1) and the TRAIL (TNF-related apoptosis inducing ligand) receptors DR4 and DRS. The bcl-2 proteins are a family of proteins involved in the response to apoptosis. Some of these proteins (such as bcl-2 and bcl-XL) are anti-apoptotic, while others (such as Bad or Bax) are pro-apoptotic. The sensitivity of cells to apoptotic stimuli can depend on the balance of pro- and anti-apoptotic bcl-2 proteins. Thus some factors for modulating apoptosis or factors that up regulate or down regulate bcl-2 proteins, modulate bcl-2 proteins, competitively inhibit such proteins, specifically behind such proteins, or active fragments thereof. Moreover, delivery of bcl-2 proteins can modulate apoptosis.

Caspases are a family of proteins that are effectors of apoptosis. The caspases exist within the cell as inactive pro-forms or zymogens. The zymogens can be cleaved to form active enzymes following the induction of apoptosis. Induction of apoptosis via death receptors results in the activation of an initiator caspase. These caspases can then activate other caspases in a cascade that leads to degradation of key cellular proteins and apoptosis. Thus some factors for modulating apoptosis are factors that up regulate or down regulate caspases, modulate caspases, competitively inhibit caspases, specifically behind caspases, or active fragments thereof. Moreover, delivery of caspases can modulate apoptosis. About 13 caspases are presently known, and are referred to as caspase-1, caspases-2, etc. Aside from the ligation of death receptors, there are other mechanisms by which the caspase cascade can be activated. For example, Granzyme B can be delivered into cells and thereby directly activate certain caspases. For example, delivery of cytochrome C can also lead to the activation of certain caspases.

An example of an apoptosis modulating factor is CK2α. CK2α potentiates apoptosis in a eukaryotic cell. CK2 biological activity may be reduced by administering to the cell an effective amount of an anti-sense stand of DNA, RNA, or siRNA. An embodiment is the use of nanoparticles to potentiate apoptosis in eukaryotic cells by decreasing the expression of casein-kinase-2. Apoptosis is inhibited or substantially decreased by preventing transcription of CK-2 DNA and/or translation of RNA. This can be carried out by introducing antisense oligonucleotides of the CK-2 sequence into cells, in which they hybridize to the CK-2 encoding mRNA sequences, preventing their further processing. It is contemplated that the antisense oligonucleotide can be introduced into the cells by introducing antisense-single stranded nucleic acid which is substantially identical to the complement of the cDNA sequence. It is also possible to inhibit expression of CK-2 by the addition of agents which degrade CK-2. Such agents include a protease or other substance which enhances CK-2 breakdown in cells. In either case, the effect is indirect, in that less CK-2 is available than would otherwise be the case.

As used herein, the term nucleic acid or polynucleotide refers to any nucleic acid molecule, including without limitation, RNA and DNA. The term encompasses sequences that include any of the known base analogs of DNA and RNA. Without limitation, the definition includes cDNA, genomic DNA, synthetic (e.g., chemically synthesized) DNA, as well as naturally-occurring and chemically modified nucleic acids, e.g., synthetic bases or alternative backbones. A nucleic acid molecule can be double-stranded or single-stranded (i.e., a sense or an antisense single strand RNA or DNA, siRNA). Polynucleotides and polynucleotide analogues (e.g., morpholinos) can be designed to hybridize to a target nucleic acid molecule. It is understood in the art that the sequence of the polynucleotide or polynucleotide analogue need not be 100% complementary to that of the target nucleic acid molecule to hybridize.

Certain embodiments provide various polypeptide sequences and/or purified polypeptides. A polypeptide refers to a chain of amino acid residues, regardless of post-translational modification (e.g., phosphorylation or glycosylation) and/or complexation with additional polypeptides, synthesis into multisubunit complexes, with nucleic acids and/or carbohydrates, or other molecules. Proteoglycans therefore also are referred to herein as polypeptides.

Anti-sense DNA compounds (e.g., oligonucleotides) treat disease, and more generally later biological activity, by interrupting cellular production of a target protein. Such compounds offer the potential benefits of 1) rational drug design rather than screening huge compound libraries and 2) a decrease in anticipated side effects due to the specificity of Watson-Crick base-pairing between the antisense molecule's sequential pattern of nucleotide bases and that of the target protein's precursor mRNA. Various antisense molecules are set forth herein. In some embodiments, the antisense molecules can be preferably targeted to hybridize to the start codon of an mRNA and to codons on either side of the start codon, e.g., within 1-20 bases of the start codon. Other codons, however, may be targeted with success, e.g., any set of codons in a sequence. The procedure for identifying additional antisense molecules will be apparent to an artisan of ordinary skill after reading this disclosure. One procedure would be to test antisense molecules of about 20 nucleic acids in a screening assay. Each proposed antisense molecule would be tested to determine its effectiveness, and the most promising candidates would form the basis for optimization. Hybridization of antisense oligonucleotides with mRNA interferes with one or more of the normal functions of mRNA, e.g., translocation of the RNA to a site of protein translation, translation of protein from the RNA, splicing of the RNA to yield one or more mRNA species, and catalytic activity which may be engaged in by the RNA. Binding of specific protein(s) to the RNA may also be interfered with by antisense oligonucleotide hybridization to the RNA. The function of a gene can be disrupted by delivery of anti-sense DNA or RNA that prevents transcription or translation of the protein encoded by the gene. This can be accomplished by providing an appropriate length oligonucleotide which is complimentary to at least a portion of the messenger RNA (mRNA) transcribed from the gene. The antisense strand hybridizes with the mRNA and targets mRNA destruction by preventing ribosomal translation, and subsequent protein synthesis. Oligonucleotides of greater length (15-30 bases) are preferred because they are more specific, and are less likely to induce toxic complications that might result from unwanted hybridization.

The incorporation of small interfering RNA (SiRNA) molecules are also contemplated, which are double stranded RNA molecules that are capable of mimicking an RNA virus infection. One advantage of using SiRNA molecules is that such molecules are very easy to design. In fact, SiRNA molecules may be based on any portion of a messenger RNA molecule or transcript and still be effective in delivering a therapeutic effect in a target cell. As an example, the casein kinase 2 mRNA transcript may be used to prepare an SiRNA molecule. Furthermore, SiRNA molecules typically have little, if any, binding issues since the SiRNA molecule need not bind to specific portion of the gene in order to be effective.

An example of a system for delivering antisense molecules is a collection of nanoparticles of less than about 50 nm loaded with CK2α and optionally made with tenascin or other cell-specific targeting molecules. Other antisense molecules, including those directed against subunits of CK2α, may alternatively be used. Shown herein, see Examples, are nanoparticles loaded with antisense CK2 used to treat an aggressive head neck carcinoma line (UM-11A) in vivo. Using a phosphodiester DNA oligomer targeted to the translation initiation site, the Applicant has shown an increase in efficacy in vitro for this embodiment as compared to liposomal antisense CK2 and cisplatin, (Unger, 2002). The Applicant has also shown a dose response against 1 mm tumor nests cultured in vitro and have shown biological activity against pilot 4 mm xenograft tumors grown in nude mice (Unger, 2002). See also Examples.

In primary human tumors tested to date (8 types), CK2 is upregulated 2 to 8 fold by kinase activity of crude homogenates or nuclear-localized protein levels suggesting a role in cell viability. Several lines of investigation support the notion that shuttling of CK2 to the nucleus (e.g. nuclear matrix and chromatin) is related to regulation of cell growth and apoptosis suppression. Rapid loss of CK2 from the nucleus is associated with cessation of cell growth, an indication of apoptosis. Prostate and SCCHN carcinoma cells appear vulnerable to antisense manipulation of CK2 protein levels. Even a modest reduction of CK2 in the nucleus resulted in extensive apoptosis. In head neck tumor biopsies, CK2 is upregulated and increased levels negatively correlate with tumor grade, stage and clinical outcome.

As shown in the Examples herein, or previously, nanoparticles of less than about 50 nm made with hydrophobic surfactants and the extracellular matrix protein tenascin selectively deliver nucleic acid cargo to solid tumors. This selective uptake is mediated by caveolar endocytosis. Nanoparticle entry into solid tumors is from the surrounding tissue (peritumoral infiltration). Local delivery via peritumoral infiltration may offer advantages over current delivery methods into solid tumors. Further increases in drug efficacy are expected to be obtained by incorporating formats exhibiting higher binding affinities for the target Protein Kinase CK2 mRNA. The effectiveness of CK2α nanoparticles was further confirmed using live mouse models, as discussed in the Examples herein.

Polynucleotide analogues or polynucleic acids are chemically modified polynucleotides or polynucleic acids. In some embodiments, polynucleotide analogues can be generated by replacing portions of the sugar-phosphate backbone of a polynucleotide with alternative functional groups. Morpholino-modified polynucleotides, referred to herein as “morpholinos,” are polynucleotide analogues in which the bases are linked by a morpholino-phosphorodiamidate backbone (See, Summerton and Weller (1997) Antisense Nuc. Acid Drug Devel. 7:187-195; and U.S. Pat. Nos. 5,142,047 and 5,185,444). In addition to morpholinos, other examples of polynucleotide analogues include analogues in which the bases are linked by a polyvinyl backbone (Pitha et al. (1970) Biochim. Biophys. Acta 204:39-48; Pitha et al. (1970) Biopolymers 9:965-977), peptide nucleic acids (PNAs) in which the bases are linked by amide bonds formed by pseudopeptide 2-aminoethyl-glycine groups (Nielsen et al. (1991) Science 254:1497-1500), analogues in which the nucleoside subunits are linked by methylphosphonate groups (Miller et al. (1979) Biochem. 18:5134-5143; Miller et al. (1980) J. Biol. Chem. 255:9659-9665), analogues in which the phosphate residues linking nucleoside subunits are replaced by phosphoroamidate groups (Froehler et al. (1988) Nucleic Acids Res. 156:4831-4839), and phosphorothioated DNAs, analogues containing sugar moieties that have 2′ O-methyl groups (Cook (1998) Antisense Medicinal Chemistry, Springer, N.Y., pp. 51-101).

Metal modification of biocompatible polymers for the purpose of enhancing particle anti-proliferative activity is applicable to any number of different delivery capsules. The metal-modified biocompatible polymers of the present invention can be incorporated in any particle geometry wherein there is a shell-like domain that comprises the particle surface. It is to be understood that within the present invention, the term metal modified may refer to any metal treatments of biocompatible polymers as disclosed in the present invention, such as, for example, with metal ions such as anions or cations, treatment with organic metals, and the like.

Nanoparticles can comprise antibodies for targeting the nanoparticles to cells or tissues, whereby bioactive or visualization agents associated with the nanoparticles may be delivered. Some embodiments include antibodies having specific binding activity for a cell recognition target, e.g., cell surface receptor, extracellular matrix molecule, growth factor receptor, or cell specific marker. Such antibodies can be useful for directing nanoparticles to specific cell types, for example.

Certain embodiments of coatings, components, and/or targets include natural and synthetic, native and modified, anionic or acidic saccharides, disaccharides, oligosaccharides, polysaccharides and glycosaminoglycans (GAGs). Dermatan sulfates, for example, have been shown to be useful for targeting molecules specifically to cells, e.g., as in U.S. Pat. No. 6,106,866. Many peptidic fragments of extracellular matrix molecules are known that are bioactive functions, e.g, the tripeptidic integrin-mediated adhesion domain of fibronectin, see also, e.g., U.S. Pat. Nos. 6,074,659 and 5,646,248. Moreover, other peptidic targeting ligands may be used, e.g., as in U.S. Pat. No. 5,846,561. Also, for example, lung targeting peptides are set forth in U.S. Pat. No. 6,174,867. Also, for example, organ targeting peptides may be used, as in U.S. Pat. No. 6,232,287. Also, for example, brain targeting peptides may be used, as in U.S. Pat. No. 6,296,832. Also, for example, heart-targeting peptides may be used, as in U.S. Pat. No. 6,303,5473.

As discussed hereinabove, the biocompatible polymer component of the present invention is a metal ion-treated biocompatible polymer component. In one embodiment, the metal-modified biocompatible polymer component is treated by a method which includes the following steps. The method steps include the step of providing a biocompatible polymer capable of being precipitated; combining the biocompatible polymer, a metal ion solution, and a precipitant; precipitating the biocompatible polymer; and resolubilizing the biocompatible polymer, forming a metal-modified biocompatible polymer.

In one embodiment, the step of combining the biocompatible polymer, a metal ion solution, and a precipitant is performed under conditions which will precipitate at least a significant portion of the biocompatible polymer. A significant portion includes about 10% of the biocompatible polymer, about 20% of the biocompatible polymer, about 30% of the biocompatible polymer, about 40% of the biocompatible polymer, about 50% of the biocompatible polymer, about 60% of the biocompatible polymer, about 70% of the biocompatible polymer, about 80% of the biocompatible polymer, about 90% of the biocompatible polymer, and about 95% of the biocompatible polymer or more.

The precipitant can be any precipitant that is known in the art to precipitate a biocompatible polymer. Where the biocompatible polymer is a protein or a polypeptide, a non-exhaustive list of precipitants includes salts, including chaotropic salts, including, without limitation, cations NH4+, K+, Na+, and Li+, and anions SO4—, HPO42—, OH—, CH3COO—, citrate, and tartrate. Precipitants also include polymers such as polyethylene glycol; alcohol; acids such as trichloroacetic acid; and the like. Methods with which to precipitate proteins using precipitants such as those mentioned herein are known in the art. In one embodiment, the precipitant is ammonium sulfate. Where the precipitant is ammonium sulfate, preferably, a saturated solution of ammonium sulfate is used in accordance with the knowledge in the art. Exact conditions of incubation, such as time of incubation, temperature, can also be determined by one of skill in the art, and include 4 degree C. and between about 4 hours and about 24 hours.

A metal ion solution according to the invention includes any metal ions including redox-active metal ions, which may have a toxic and/or anti-proliferative effect on cells, particularly hyperproliferative cells, in vitro or in vivo. Included are metal ions that are known or are theorized in the art to enhance oxidative stress in a cell. Without being bound by theory, the inventor believes that the inclusion of metal ions into the biocompatible polymer component of the particle may enhance the anti-proliferative effects of any bioactive agent included in the nanocapsule (e.g., the cargo). It is also contemplated that incorporation of metal ions into the biocompatible polymer to effect anti-proliferative activity in the absence of bioactive agents cargo.

Oxidative stress is a major component of inflammation and may cause damage to biological systems, including, without limitation, cells and/or tissues that are the targets of the particles of the present invention. Some agents causing oxidative stress, termed RONS (reactive oxygen and nitrogen species), include superoxide (O2—), hydrogen peroxide (H2O2), hydroxyl radical (OH) and peroxynitrite (ONOO—), among others. Numerous studies have demonstrated that RONS initiate and/or perpetuate the lipid peroxidation process, degrade DNA, destroy endothelial cells and induce increased vascular permeability. Redox-active metal ions are capable of mediating oxidative damage as will be recognized by the one skilled in the art. In addition, they may enhance ONOO—mediated damage, resulting in enhanced nitration of aromatics. Metal ion-activated decomposition of ONOO— is condition-specific and can generate varying amounts of OH and nitrated aromatics. As used in herein, the terms metal and metal composition are interchangeable and refer to inorganic or organic species. In one embodiment, the metal is an inorganic species. Appropriate metal ions and/or redox-active metal ions are known in the art, and include heavy metals, such as, ions of iron, barium, copper, zinc, tin, gadolinium, vanadium, chromium, molybdenum, arsenic, selenium, beryllium, manganese, cobalt, nickel, cadmium, mercury, and lead, and combinations thereof, to form the metal ion solution. In one embodiment, the metal ion solution comprises at least one cation selected from the group consisting of arsenic cations, iron cations, manganese cations, selenium cations, molybdenum cations, mercury cations, and combinations thereof. The metal ion solution, in one embodiment, comprises metal ions As3+, Se4+, Hg2+, and Mo5+. Preferably, the metal ions are pharmaceutically acceptable. In addition to simple metal salts, one skilled in the art will recognize that appropriate metal ions may be contained within organometallo compounds of the configuration R-metal-R, R-metal-H, or R-metal-X.

Appropriate amounts of metal ions to use can be selected by one of skill in the art, taking into consideration concentrations known to cause oxidative stress in cells, toxicity of the metals, and so on. In one embodiment, the metal ion solution comprises the at least one cation in a concentration of between about 0.1 part per billion (ppb) and 1 part per thousand (ppt). Appropriate concentrations of each metal ion include about 0.1 ppb, 0.5 ppb, 1 ppb, about 5 ppb, about 10 ppb, about 15 ppb, about 20 ppb, about 25 ppb, about 30 ppb, about 35 ppb, about 40 ppb, about 50 ppb, about 100 ppb, about 150 ppb, about 200 ppb, about 250 ppb, about 300 ppb, about 350 ppb, about 400 ppb, about 450 ppb, about 500 ppb, about 550 ppb, about 600 ppb, about 650 ppb, about 700 ppb, about 750 ppb, about 800 ppb, about 850 ppb, about 900 ppb, about 950 ppb, about 1 part per million (ppm), about 5 ppm, about 10 ppm, about 15 ppm, about 25 ppm, about 30 ppm, about 35 ppm, about 40 ppm, about 45 ppm, about 50 ppm, about 75 ppm, about 100 ppm, about 150 ppm, about 200 ppm, about 250 ppm, about 300 ppm, about 350 ppm, about 400 ppm, about 450 ppm, about 500 ppm, about 550 ppm, about 600 ppm, about 650 ppm, about 700 ppm, about 750 ppm, about 800 ppm, about 850 ppm, about 900 ppm, about 950 ppm, and about 1 ppt. In one embodiment, the metal ion solution comprises As3+ at about 250 parts per billion (ppb), Se4+ at about 25 parts per million (ppm), Hg2+ at about 2.5 ppm, and Mo5+ at about 25 ppm. In one embodiment, the metal ion solution comprises a total metal ion concentration optionally not exceeding 2 parts per thousand, optionally not exceeding 5 parts per thousand, optionally not exceeding 10 parts per thousand.

Any concentration of biocompatible polymer which is compatible with the metal modification method as disclosed herein may be utilized in the inventive processes. In one embodiment, the biocompatible polymer is a polypeptide, and the concentration of polypeptide is between about a 0.01 mg/ml solution of the polypeptide and about a 100 mg/ml solution of the polypeptide. In another embodiment, concentration of polypeptide is between about a 0.1 mg/ml solution of the polypeptide and about a 10 mg/ml solution of the polypeptide. In another embodiment, concentration of polypeptide is between about a 0.1 mg/ml solution of the polypeptide and about a 1 mg/ml solution of the polypeptide. Appropriate concentrations may be chosen by one of skill in the art in accordance with variables such as solubility of the biocompatible polymer, susceptibility to the particular precipitant, and convenience and scale-up factors, and other factors as are known in the art.

As used herein, the terms oligopeptide, peptide, polypeptide, and protein are interchangeable and refer to a polymer of amino acid residues. The terms apply to amino acid polymers in which one or more amino acid residue is an artificial chemical mimetic of a corresponding naturally occurring amino acid, as well as to naturally occurring amino acid polymers and nonnaturally occurring amino acid polymer. These terms also encompass the term antibody. Antibodies, which may be generated by methods known in the art, e.g., recombinantly, or via hybridoma processes. Such antibodies can be of any immunoglobulin class, including IgM, IgG, IgE, IgA, IgD, and any subclass thereof and include chimeric antibodies as well as intact molecules or fragments thereof that are capable of binding to an epitope. The term “epitope” refers to an antigenic determinant on an antigen to which an antibody binds. The terms antibody and antibodies include polyclonal antibodies, monoclonal antibodies, humanized or chimeric antibodies, single chain Fv antibody fragments, Fab fragments, and F(ab)₂ fragments. Optionally an antibody is an internalizing antibody.

According to the present invention, the metal ions are associated with the biocompatible polymer in any manner, including, without limitation, covalent interactions or non covalent interactions. Non-covalent interactions include salt bridges, Van der Waals interactions, and the like. In one embodiment, the metal ion is associated with the biocompatible polymer in a non-covalent manner. Without being bound by theory, the inventor believes that the association between the metal ion(s) and the biocompatible polymer is non covalent. However, the exact nature of the association between the metal ion(s) and the biocompatible polymer is not critical to the present invention. In one embodiment, the association between the bioactive component and the surfactant is non-covalent, and the association between the metal-ion treated biocompatible polymer and the coated bioactive component is non-covalent.

In one embodiment, tenfibgen (a domain of tenascin) or tenascin polypeptides (as the biocompatible polymer) are precipitated from cell culture supernatants using metal ion solution-containing ammonium sulfate such that the metal ions of the present invention known to promote oxidative stress are adsorbed onto or otherwise associated with the biocompatible polymer. Metal ion-treated tenfibgen (a domain of tenascin) or tenascin polypeptides, for example, may be prepared with or treated with pharmaceutically acceptable heavy metals by precipitating in, in one embodiment, a saturated ammonium sulfate solution prepared with a metal ion of the present invention. In one embodiment, incubation of about a 0.1-1 mg/ml solution of a protein such as tenfibgen (a domain of tenascin) or tenascin polypeptides, in a ratio of 1:2 with a saturated ammonium sulfate solution, is performed for about 4-24 hours before recovering metal ion-treated biocompatible polymer (also known as modified coating ligand) by centrifugation. As discussed above, metal concentrations in the ultrapure ammonium sulfate may range from 1 ppb to 1 ppt.

In one embodiment, the metal-ion treated biocompatible polymers are incorporated into and/or associated with a coated bioactive agent of the present invention. In other embodiments, the metal ion-treated biocompatible polymers are incorporated into and/or associated with a pharmaceutical formulation, including an encapsulated pharmaceutical agent. Any system of encapsulated pharmaceutical agent is compatible with the metal ion-treated polymers of the present invention, and include such encapsulation systems as polymer-based drug-delivery systems developed as microspheres for injection, implants, transdermal patches, and aerosols for inhalation (Domb et al., Handbook of Biodegradable Polymers, Harwood Academic Publishers, Amsterdam, 1997; Putney et al., Nature Biotechnology 16: 153-157, 1998; Edwards et al., Science 276: 1868-1871, 1997); lipid-based drug delivery systems have been developed as unilamellar, multilamellar (Gregoriadis, Liposome Technology, Vols. I, II, III, CRC Press, Boca Raton, Fla., 1993), and multivesicular liposomes (U.S. Pat. No. 5,422,120 to Kim; U.S. Pat. No. 5,723,147 to Kim et al). Other appropriate systems are protein based and include Kumar et al., Nature Vol. 448 pp. 39-43 (2007).

The present inventive metal ion treatment of biocompatible polymers provides a number of surprising results. For example, the metal ion treatment is performed on the targeting ligand, not on the bioactive agent, of the particles, yet increased efficacy (i.e., increased antiproliferative effect) of the bioactive agent was observed. The targeting ligand (i.e., a biocompatible polymer) was not previously contemplated to potentially contribute to the anti-proliferative effects of the particles of the invention (comprising, e.g., antiproliferative bioactive agents). Further, the concentrations of metal ion(s) in the metal ion solution used in the treatment step, for the most part, are concentrations that would not be expected to exert any anti-proliferative effects. Thus, clearly there is greater than an additive effect, (e.g., a synergistic effect) of the metal ion treatment of the biocompatible polymers of the present invention and the bioactive agent, on proliferative cells. See, e.g., Examples 1-7.

The formulation of therapeutic compositions of the present invention, and their subsequent administration is believed to be within the skill of those in the art. In general, for therapeutics, a patient in need of such therapy is administered a compound in accordance with the invention, commonly in a pharmaceutically acceptable carrier, in dosages and novel regimen strategies as described elsewhere herein. In one embodiment of the present invention, administration is determined per kg of body weight depending on the age of the patient and the severity of the disease state being treated.

In one embodiment, the metal-modified biocompatible polymer is delivered at a dose of total metals of less than about 10 ng/kg body weight. In other embodiments, the metal-modified biocompatible polymer is delivered at a dose of total metals of less than about 1 ng/kg body weight, less than about 100 nanogram(ng)/kg body weight, less than about 10 ng/kg body weight, less than about 1 ng/kg body weight less than about 100 picogram(pg)/kg body weight, less than about 10 pg/kg body weight, less than about 1 pg/kg body weight less than about 100 femtogram(fg)/kg body weight, less than about 10 fg/kg body weight, less than about 1 fg/kg body weight less than about 100 attogram(ag)/kg body weight, less than about 10 ag/kg body weight, less than about 1 ag/kg body weight.

In another embodiment, the metal-modified biocompatible polymer is delivered at a dose of total metals of between about 1 ng/kg body weight and about 1 attog/kg body weight. In other embodiments, dosages are between 100 ng/kg body weight and about 10 ag/kg body weight. In other embodiments, dosages are between about 10 nanogram(ng)/kg body weight and about 100 attogram(ag)/kg body weight. Zeptogram dosing is also possible. In other embodiments, dosages are between about 1 ng/kg body weight and about 1 fg/kg body weight. In another embodiment, dosages are between about 100 picogram(pg)/kg body weight and about 10 femtogram(fg)/kg body weight. In other embodiments, dosages are between about 10 pg/kg body weight and about 100 fg/kg body weight. In other embodiments, dosages are between about 1 pg/kg body weight and about 10 fg/kg body weight.

In some embodiments, the metal-modified biocompatible polymer is formulated as a pharmaceutical composition which contains a pharmaceutically acceptable carrier. Such a pharmaceutically acceptable carrier includes, for example, the sub-50 nanocapsules as described elsewhere herein.

Further, the treatment regimen may last for a period of time which will vary depending upon the nature of the particular disease, its severity and the overall condition of the patient, and may extend from once daily to once every 20 years. Following treatment, the patient is monitored for changes in his/her condition and for alleviation of the symptoms of the disease state. The dosage of the compound may either be increased in the event the patient does not respond significantly to current dosage levels, or the dose may be decreased if an alleviation of the symptoms of the disease state is observed, or if the disease state has been ablated.

In some cases it may be more effective to treat a patient with a compound of the invention in conjunction with other traditional therapeutic modalities. For example, a patient being treated for a viral disease may be administered a compound of the invention in conjunction with a known antiviral agent, or a patient with atherosclerosis may be treated with a compound of the invention following angioplasty to prevent reocclusion of the treated arteries.

Following successful treatment, it may be desirable to have the patient undergo maintenance therapy to prevent the recurrence of the disease state, wherein the compound of the invention is administered in maintenance doses, ranging from 0.01 zeptogram to 100 mcg per kg of body weight of total metals derived from metal-modified biocompatible polymer, once or more daily, to once every 20 years.

The pharmaceutical compositions of the present invention may be administered in a number of ways depending upon whether local or systemic treatment is desired and upon the area to be treated. Administration may be topical (including ophthalmic, vaginal, rectal, intranasal, transdermal, transtymphanic, transmucosal, buccal), oral or parenteral. Parenteral administration includes intravenous administration, subcutaneous, intraperitoneal, intra-ocular, intratumoral, intra urethral, intra hepatic or intramuscular injection, or intrathecal or intraventricular administration.

Formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Compositions for oral administration include powders or granules, suspensions or solutions in water or non-aqueous media, capsules, sachets or tablets. Thickeners, flavoring agents, diluents, emulsifiers, dispersing aids or binders may be desirable.

Compositions for intrathecal or intraventricular administration may include sterile aqueous solutions which may also contain buffers, diluents and other suitable additives.

Formulations for parenteral administration may include sterile aqueous solutions which may also contain buffers, diluents and other suitable additives.

Dosing is dependent on severity and responsiveness of the disease condition to be treated, with the course of treatment lasting from several days to several months, or until a cure is effected or a diminution of disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual compounds, and can generally be estimated based on EC50 found to be effective in in vitro and in vivo animal models. Dosages may be given once or more daily, weekly, monthly or yearly, or even once every 2 to 20 years.

In one embodiment, the present invention is useful for treating any condition in which inhibiting a target gene is potentially of use. In one embodiment, the present invention may be used for treating a proliferative disease. By “proliferative disease” is meant any human or animal disease or disorder, affecting any one or any combination of organs, cavities, or body parts, which is characterized by single or multiple local abnormal proliferations of cells, groups of cells, or tissues, whether benign or malignant. There are many disorders associated with a dysregulation of cellular proliferation. The conditions of interest include, but are not limited to, the following conditions. In one embodiment, proliferative disease includes proliferation and/or migration of smooth muscle cells, and/or inflammatory cells into the intimal layer of a vessel, resulting in restricted blood flow through that vessel, i.e. neointimal occlusive lesions. Occlusive vascular conditions of interest include atherosclerosis, graft coronary vascular disease after transplantation, vein graft stenosis, peri-anastomatic prosthetic graft stenosis, restenosis after angioplasty or stent placement, and the like. Other proliferative diseases include abnormal angiogenesis, notably tumor growth(including tumor nests) and metastasis, and other conditions in which blood vessel proliferation is increased, such as psoriasis and arthropathies. Other proliferative diseases include those where there is hyperproliferation and tissue remodelling or repair of reproductive tissue, e.g. uterine, testicular and ovarian carcinomas, endometriosis, squamous and glandular epithelial carcinomas of the cervix, etc. Proliferative diseases include cirrhosis of the liver (a condition in which scarring has overtaken normal liver regeneration processes), treatment or inhibition of keloid (hypertrophic scar) formation (disfiguring of the skin in which the scarring process interferes with normal renewal), diabetic retinopathy, psoriasis (a common skin condition characterized by excessive proliferation of the skin and delay in proper cell fate determination), benign tumors, fibrocystic conditions, and tissue hypertrophy (e.g., prostatic hyperplasia).

Examples of proliferative diseases, disorders, and/or conditions that can be treated, prevented, and/or diagnosed by the particles of the present invention include, but are not limited to neoplasms located in the: colon, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital. Similarly, other hyperproliferative diseases, disorders, and/or conditions can also be treated, prevented, and/or diagnosed by particles of the present invention. Examples of such hyperproliferative diseases, disorders, and/or conditions include, but are not limited to: hypergammaglobulinemia, lymphoproliferative diseases, disorders, and/or conditions, paraproteinemias, purpura, sarcoidosis, Sezary Syndrome, Waldenstron's Macroglobulinemia, Gaucher's Disease, histiocytosis, and any other hyperproliferative disease, besides neoplasia, located in an organ system listed above.

The present invention is more particularly described in the following Examples which are intended as illustrations only since numerous modifications and variations within the scope of the present invention will be apparent to those skilled in the art.

### EXAMPLES

### Example 1

### Preparation of Tumor-Targeted Nanoparticles

This example describes how colloidal formulations of diverse cargos and biocompatible polymers may be generated. Nanoparticles for uptake, biodistribution and efficacy studies were prepared by the “dispersion atomization” method described in U.S. Pat. No. 6,632,671, which is incorporated herein by reference in its entirety, with some modifications.

Tenascin (“TN”) is an extracellular matrix molecule that is useful for nanoparticles as a biocompatible polymer and/or as a targeting moiety. Tenascin is a branched, 225 KD fibronectin-like (FN) extracellular protein prominent in specialized embryonic tissues, wound healing and tumors. The appearance of tenascin-C surrounding oral squamous cell carcinomas appears to be a universal feature of these tumors, while tenascin-rich stroma has been consistently observed adjacent to basal cell, esophageal, gastric, hepatic, colonic, glial and pancreatic tumor nests. Production of TN by breast carcinoma cells and stromal fibroblasts correlates with increased invasiveness. In the adult, normal cells aside from wound-activated keratinocytes, do not migrate on tenascin. However, integrin receptors capable of mediating migration on TN by carcinoma cells include a2B1, αvβ3 and αvβ6. In one embodiment, TN nanoparticles deliver nucleic acids specifically via receptor-mediated caveolar endocytosis. TN, or any subdomain(s) thereof, are suitable cell recognition polypeptides according to the invention.

Tenascin has been implicated in cancer activities and also as being specific for smooth muscle cells; furthermore, peptidic domains of tenascin have been identified e.g., as in U.S. Pat. No. 6,124,260, and are known in the art. In one embodiment, tenascin suitable for the present invention is H. sapiens tenascin C, Genbank Accession No. NM_{—}002160. Moreover, tenascin peptides and domains for adhesion with particular cell types, as well as functional and structural aspects of tenascin, have been disclosed and are known in the art, e.g., Aukhill et al., J. Biol. Chem., Vol. 268, No. 4, 2542-2553. Tenascin and/or any of its domains are suitable for the present invention. In one embodiment, the fibrinogen fragment of tenascin (also referred to herein as Fbg-L domain of tenascin-C or tenfibgen or TBG (nucleotide sequence of tenfibgen follows:) (ATTGGACTCCTGTACCCCTTCCCCAAGGACTGCTCCCAAGCAATGCTGA ATGGAGACACGACCTCTGGCCTCTACACCATTTATCTGAATGGTGATAAGGCTCA GGCGCTGGAAGTCTTCTGTGACATGACCTCTGATGGGGGTGGATGGATTGTGTTC CTGAGACGCAAAAACGGACGCGAGAACTTCTACCAAAACTGGAAGGCATATGCT GCTGGATTTGGGGACCGCAGAGAAGAATTCTGGCTTGGGCTGGACAACCTGAAC AAAATCACAGCCCAGGGGCAGTACGAGCTCCGGGTGGACCTGCGGGACCATGGG GAGACAGCCTTTGCTGTCTATGACAAGTTCAGCGTGGGAGATGCCAAGACTCGCT ACAAGCTGAAGGTGGAGGGGTACAGTGGGACAGCAGGTGACTCCATGGCCTACC ACAATGGCAGATCCTTCTCCACCTTTGACAAGGACACAGATTCAGCCATCACCAA CTGTGCTCTGTCCTACAAAGGGGCTTTCTGGTACAGGAACTGTCACCGTGTCAAC CTGATGGGGAGATATGGGGACAATAACCACAGTCAGGGCGTTAACTGGTTCCAC TGGAAGGGCCACGAACACTCAATCCAGTTTGCTGAGATGAAGCTGAGACCAAGC AACTTCAGAAATCTTGAAGGCAGGCGCAAACGGGCATAA) is used as the biocompatible polymer and/or the cell recognition polypeptide. Tenascin, its subdomains, or any other biocompatible polymer may be expressed or produced by methods known in the art. The interaction between smooth muscle cells and the Fbg-L domain of tenascin-C is involved in cell adhesion and migration, and blocking this interaction would blunt SMC migration from media into the neointima and thereby affect neointimal formation, see LaFleur et al., J. Biol. Chem., 272(52): 32798-32803, 1997. Further, cardiac myocyte activity involved tenascin, e.g., Yamamoto et al., J. Biol. Chem., (274) 31: 21840-21846, 1999.

Hyaluronan is also an extracellular matrix molecule that is useful for nanoparticles. Hyaluronan is preferentially expressed by hepatocytes and has been implicated angiogenesis and reactive tumor stroma, particularly in prostatic tumors, see Tuxhorn et. al, J Urol 166: 2472-2483, 2001. It is available in a variety of forms and has many known uses, e.g., as in U.S. Pat. No. 5,902,795.

Diverse cargos are suitable for particle formulation involving metal-modified biocompatible polymers including inhibitors or cell survival and stress response pathways such as oligonucleotides inhibition production of critical enzymes such as Casein Kinase 2, modulators of protein-protein binding BCL-2 and BCL-XL or transcription factors such as NFKappa-B without wishing to be bound by theory. Combination of a stressor such as a metal load inducing focal reactive oxygen species with inhibition of cellular survival and/or stress responses will provide maximum cytotoxic activity with minimal dosing, limiting the possibility of ancillary damage to non-target or bystander tissue. In another embodiment, particle formulation involving metal-modified polymers can be combined with agents acting through non-ROS mechanisms (e.g. microtubule inhibitors) or effective agents limited by transport deficiencies (e.g. multi-drug resistance).

Briefly, to prepare each formula below, the following procedures were used:

Formula A, 250 μg of a model iodosiRNA against Red Fluorescent Protein (full substitution of IdU and IdA was attempted for adenine and uridines on guide or antisense strand) was first complexed with 87.5 μg of spermine (Sigma Chemical Co., St. Louis, Mo.), and dispersed into 150 μl of sterile water using a water-insoluble surfactant system (2, 4, 7, 9-tetramethyl-5-decyn-4,7-diol (TM-diol), 6.25 μg in DMSO or SE-30 (Air Products)). The iodine-derivatized siRNA used in this formula was loaded with about 1% iodine by weight as measured by neutron activation analysis (NAA). Following emulsification with a water-miscible solvent (DMSO), the complexes were then inverted and diluted by the addition of 750 μl of PBS.

The resultant hydrophobic micelles were coated (non-covalently) by the addition of 12.5 μg of recombinant fibrinogen fragment of tenascin (TBG; prepared by the method of Aukhill et al. (1993, J. Biol. Chem., 268:2542-53) then atomized into a modified LiCl salt receiving solution (135 mM Li⁺, 13.75 mM Ca²⁺, 18 nM Sr²⁺, 3.5 nM Mg²⁺ (all ultrapure)). Following cold-room incubation (4° C.) with nominal rotation in 50 ml round-bottomed tubes for 48 hours, which stabilizes the coated micelles in the salt solution, the sub-50 nm nanocapsules were recovered by centrifugation at 20,000×g for 2 hrs and resuspended in PBS+10% lactitol (at a concentration of 0.5 μg/μl) for filter sterilization through a 0.2 μm filter. In all formulations described, a small amount (1% of coating weight) of Syrian Hamster IgG was “spiked” into the ligand coat to enable immunodetection of nanocapsules uptake by anti-syrian hamster IgG antibodies. Average capsule size was less than 50 nm as measured by tapping mode atomic force microscopy using average elliptical diameters of a 1 ng/ml sample dried down on a mica sheet.

Tenfibgen (TBG) preparation: For Formulas B-H, TBG was prepared by the method of Aukhil with modifications, i.e. TBG was isolated and refolded from bacterial lysate by washing the insoluble pellet once with lysis buffer (50 mM Tris-HCl, 1.0 mM EDTA, 0.1 M NaCl, 0.2 mg/ml lysozyme, 0.1% Triton X-100, 0.1 mM PMSF, pH 8.0), containing 2 M urea and resuspending in 4M GuCL, 5 mM DTT in 0.02 M Tris-HCl, pH 8.0. After additional centrifugation, the clarified TBG solution was diluted with 2 M Guanidine-HCl, 20 mM Tris-HCl, pH 8.0 to make a final OD280 of about 1 and diluted dropwise about 10-fold into 20 mM Tris-HCl, 0.2 M NaCl, 0.5 M Arginine-HCl, 10 uM CuCl2 pH 8.0 for overnight stirred incubation (4° C.). After diafiltration against 20 mM Tris-HCl, pH 8.0 with an approximate 4-5 fold reduction in concentration and 0.45 uM filtration, a final purification was performed on heparan sepharose in 20 mM Tris-HCl, pH 8.0, with elution by bringing the NaCl concentration to 0.6 M.

Formula B: sub-50 nm nanocapsules coated with TBG were generated as described in Formula A except that 6.3 mcg of TBG (reprecipitated in ultra-pure 40% ammonium sulfate containing 250 ppb As⁺³, 25 ppm Se⁺⁴, 2.5 ppm Hg⁺² and 25 ppm Mo⁺⁵ for about 16 hours) was added to 500 mcg of a small molecule inhibitor to CK2 (DMAT, Sigma). When generating these nanocapsules, the Tbg-coated micelles were atomized into a modified LiCl salt receiving solution (70 mM Li⁺, 14 mM Ca²⁺, 37.5 nM Sr²⁺, 12.5 nM Mg²⁺ (all ultrapure)) and capsules were incubated for 14.5 hours before centrifugation. Capsules were resuspended following centrifugation in PBS+10% Lactitol. Average capsule size was less than 50 nm as measured by tapping mode atomic force microscopy using elliptical diameters of a 1 ng/ml sample dried down on a mica sheet.

Formula C: sub-50 nm nanocapsules coated with TBG were generated as described in Formula A except that 31.25 mcg of TBG (not metal-modified) was added to 500 mcg of an antisense oligo to CK2 (phosphodiester 3′ and propylendblocked-20ME RNA chimeric, “LCK-6”, PCT/US 2005/045820) and condensed with 125 mcg of 10 kD polyornthine (Sigma). When generating these nanocapsules, the Tbg-coated micelles were atomized into a modified LiCl salt receiving solution (135 mM Li⁺, 9 mM Ca²⁺, 7.5 nM Sr²⁺, 2.3 nM Mg²⁺ (all ultrapure)) and capsules were incubated for 48 hours before centrifugation. Capsules were resuspended following centrifugation in PBS+10% Lactitol. Average capsule size was less than 50 nm as measured by tapping mode atomic force microscopy using elliptical diameters of a 1 ng/ml sample dried down on a mica sheet.

Formula D: sub-50 nm nanocapsules coated with TBG were generated as described in Formula A except that 31.25 mcg of TBG (precipitated in ultra-pure 40% ammonium sulfate containing 250 ppb As⁺³, 25 ppm Se⁺⁴, 2.5 ppm Hg⁺² and 25 ppm Mo⁺⁵ for about 16 hours) was added to 500 mcg of an antisense oligo to CK2 (phosphodiester 3′ and propylendblocked-20ME RNA chimeric, “LCK-6”, PCT/US 2005/045820) and condensed with 125 mcg of 10 kD polyornthine (Sigma). When generating these nanocapsules, the Tbg-coated micelles were atomized into a modified LiCl salt receiving solution (135 mM Li⁺, 9 mM Ca²⁺, 7.5 nM Sr²⁺, 2.3 nM Mg²⁺ (all ultrapure)) and capsules were incubated for 48 hours before centrifugation. Capsules were resuspended following centrifugation in PBS+10% Lactitol. Average capsule size was less than 50 nm as measured by tapping mode atomic force microscopy using elliptical diameters of a 1 ng/ml sample dried down on a mica sheet and a surface charge of −6±7.5 mev was measured on Zetasizer 4 dynamic light scattering device at a potential of 20 volts with a 2-second pause between measurements in 1 mM KCl at 2 μg/ml.

Formula E: sub-50 nm nanocapsules coated with TBG are generated as described in Formula A except that 25 mcg of TBG (reprecipitated in ultra-pure 40% ammonium sulfate containing 250 ppb As⁺³, 25 ppm Se⁺⁴, 2.5 ppm Hg⁺² and 25 ppm Mo⁺⁵ for about 26 hours) are added to 500 mcg of a small molecule inhibitor of microtubules, Paclitaxel (Sigma). When generating these nanocapsules, the Tbg-coated micelles are atomized into a modified LiCl salt receiving solution (135 mM Li⁺, 9 mM Ca²⁺, 18 nM Sr²⁺, 5 nM Mg²⁺ (all ultrapure)) and capsules are incubated for 48 hours before centrifugation. Capsules are resuspended following centrifugation in PBS+10% Lactitol. Average capsule size is less than 50 nm as measured by tapping mode atomic force microscopy using elliptical diameters of a 1 ng/ml sample dried down on a mica sheet.

Formula F: sub-50 nm nanocapsules are generated as described in Formula A except that 25 mcg of Hyaluronan, recombinant, 1 mM kD (Lifecore Biomedical, precipitated in ultra-pure 40% ammonium sulfate containing 250 ppb As⁺³, 25 ppm Se⁺⁴, 2.5 ppm Hg⁺² and 25 ppm Mo⁺⁵ 5 for about 16 hours) is added to 500 mcg of an antisense oligo to BCL-2/BCL-xL (5′-AAGGCATCCCAGCCTCCGTT-3′, JNCI (2001) 93:463-471). When generating these nanocapsules, the hyaluronan-coated micelles are atomized into a modified LiCl salt receiving solution (135 mM Li⁺, 9 mM Ca²⁺, 13.75 nM Sr²⁺, 2.5 nM Mg²⁺ (all ultrapure)) and capsules are incubated for 48 hours before centrifugation. Capsules are resuspended following centrifugation in PBS+10% Lactitol. Average capsule size are less than 50 nm as measured by tapping mode atomic force microscopy using elliptical diameters of a 1 ng/ml sample dried down on a mica sheet.

Formula G1: sub-50 nm nanocapsules coated with TBG were generated as described in Formula A for in vitro testing except that 6.3 mcg of TBG (not metal-modified) was added to 500 mcg of a phosphodiester antisense oligo to CK2alpha (phosphodiester, “asCK2”, PCT/US 2005/045820) and condensed with 125 mcg of 10 kD polyornthine (Sigma). When generating these nanocapsules, the Tbg-coated micelles were atomized into a LiCl and CaCl2 salt receiving solution (200 mM Li⁺, 10 mM Ca2⁺) and capsules were incubated for 13 hours before centrifugation. Capsules were resuspended following centrifugation in PBS+10% glucose or sorbitol. Average capsule size was less than 50 nm as measured by tapping mode atomic force microscopy using elliptical diameters of a 1 ng/ml sample dried down on a mica sheet. Other in vitro capsule were very similar with the following minor changes; (G2) TBG was his-tagged and particles were incubated for 14.5 hours, (G3) TBG was his-tagged and precipitated in 250 ppb As⁺⁴ and 25 ppm Se⁺⁴ and particles were incubated for 14.5 hours in modified LiCl salt receiving solution (70 mM Li⁺, 14 mM Ca²⁺,100 nM Mg²⁺ (all ultrapure))-surface charge was −1.1±3.4 mev, (G4) TBG was his-tagged and precipitated in 25 ppm As⁺³, 12.5 ppm Se⁺⁴ and 50 ppb Pb⁺² and particles were incubated for 14.5 hours modified LiCl salt receiving solution (70 mM Li⁺, 14 mM Ca²⁺, 2 nM Sr²⁺, 100 nM Mg²⁺ (all ultrapure))-surface charge was 0.3-±2.2, (G5) TBG was his-tagged and precipitated in 250 ppb As⁺³, 25 ppm Se⁺⁴, 2.5 ppm Hg⁺² and 2.5 ppm Mo⁺⁵ and particles were incubated for 14.5 hours modified LiCl salt receiving solution (70 mM Li⁺, 14 mM Ca²⁺, 100 nM Mg²⁺ (all ultrapure)).

### Example 2

### Targeting of Primary and Metastatic Tumor Burden with Specific, Tumor-Targeted Nanoparticles in Human Xenograft Tumors

The specificity of site-directed targeting of nanoparticles for intracellular uptake to tumors and micrometastatases was investigated by treating mice bearing SSCHN (squamous cell carcinoma of the head and neck, FaDu) xenograft tumors with TBG nanoparticles containing iodine-derivatized siRNA against Red Fluororescent Protein (RFP, Example 1 Formula A).

TBG is useful as a cell recognition component in a tumor-targeting nanoparticle as is Tenascin-C, from which it is derived. Besides being consistently observed in stroma adjacent to many solid tumors, Tenascin has also been linked to the vascularization of tumor tissue; specifically, tenascin (i) has been found in and around tumor microvessels, (ii) is produced by migrating endothelial cells, and (iii) when coated on tissue culture plates, stimulates sprouting by and migration of endothelial cell. Antibodies to tenascin were one of the earliest anti-angiogenic approaches explored in cancer treatment, but never worked well enough to move beyond early clinical development. Early enthusiasm for a potential therapeutic uses for tenascin in oncology waned as knock-out mice studies failed to confirm any critical role in tumor biology. Despite these unfavorable precedents, we tested the biodistribution of tenfibgen s50 nanoparticles in tumor-bearing mice.

Surprisingly, however, as described below, we found the TBG s50 nanoparticle provides specific delivery of labeled model cargo to not only primary but metastatic tumor, which showed that s50 nanoparticles can be used for therapeutic delivery of biologics to proliferating tumor cells and associated tumor-derived microvasculature wherever they are located.

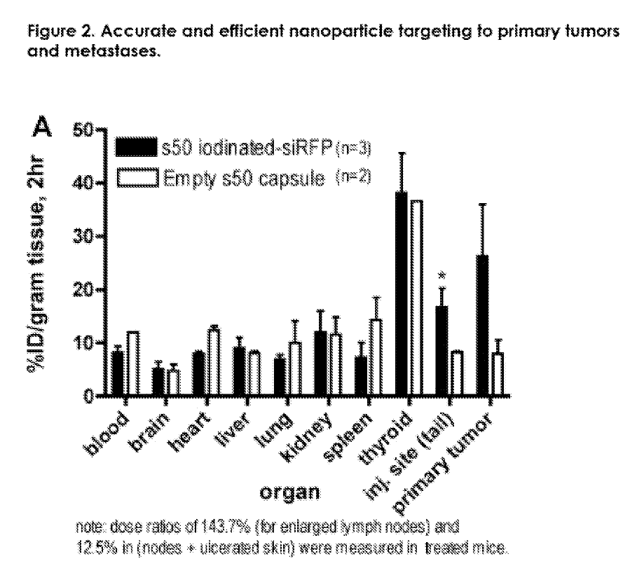

To test the biodistribution of tenfibgen nanoparticles, we administered tenfibgen nanocapsules containing either iodinated siRNA or sugar to nude mice bearing small, flank FaDu tumors (50-150 mg). Mice received an intravenous dose by tail vein of 200 ug of siRNA or about 8800 counts and were euthanized 2 hours later. Major organs, tumor and blood were collected and sent to a nuclear reactor facility for counting of biodistribution by neutron activation analysis of Iodine-128 (See FIG. 2). As expected iodine levels were the highest in thyroid, but not different between sugar and iodosiRNA indicating that iodosiRNA was not accumulating in thyroid. Relative to endogenous iodine background in sugar capsule mice, dose ratios indicated the highest levels of iodosiRNA were in the injection site (˜8.5% net) and primary tumor (˜15.3% net). No accumulation was detected in the RES organs of liver, lung or spleen (˜0 to 1% net). These data indicate that off-target toxicity common with chemotherapy drugs will not occur with accurate particle delivery and that surprisingly, the holy grail of efficient and specific tumor delivery is possible.

Dose ratios above average background were detected in metastases (144(enlarged lymph node) and 12.5% (ulcerated skin)). Lymph nodes are very difficult to dissect in non-tumor bearing mice but readily apparent post metastasis in these metastatic HNC xenograft models. The measuring of a dose ratio above 100% (high percentage of counts divided by low tissue weight) suggests that well vascularized lesions (such as a lymph node) may accumulate nanocapsules before a more poorly vascularized primary tumor. In follow-up microscopy of NAA FaDu tumor retains, we immunodetected capsules in primary tumors by performing confocal microscopy for Syrian Hamster IgG “spiked” into the particle coating. Microscopy shows 1) abundant punctate particle immunosignal colocalized with a lattice-like signal denoting Keratin-14(+) cytoplasm of the tumor cells in treated tumor and 2) no punctate signal in untreated tumor. Capsule accumulation also occurs in nuclear regions which are represented by “black holes” surrounded by cytoplasmic K-14 signal. These results confirm that therapeutic cargo accumulation in tumors was mediated by intact capsules so that iodine signal represents capsule delivery and not “free drug” post-dissolution. We also immunodetected capsules as punctate Syrian Hamster IgG-(+) signal in dermal metastases at 2 hr (from 12% ulcerated skin sample) supporting the notion that tumor-specific targeting to well-vascularized small metastases may occur before primary tumor. We concluded tenfibgen nanoparticles can deliver therapeutic loads accurately and efficiently to primary and distant disease.

### Example 3

### Targeted S50 Nanoparticle Increases Cellular Exposure for Hydrophobic Small Molecules

To investigate whether sub-50 nm colloidal delivery could significantly enhance delivery of challenging pharmaceuticals, we undertook formulation of a poorly, water-soluble small molecule inhibitor of CK2, DMAT for in vitro studies (prepared as in Example 1, Formula B). An initial comparison of free to formulated DMAT was carried out by comparing 48 hour survival of androgen-resistant PC-3 prostate carcinoma cells plated on 3-D synthetic matrices in 96 wells (Corning Ultramax) to promote caveolar development at cell surfaces. s50 ligand-directed nanoparticles are believed to more efficiently enter cells through non-clathrin-mediated processes such as caveolae, thus avoiding lysosomal sequestration common to other forms of delivery. Cells received a series of single doses and survival was assayed by thymidine incorporation by pulsing in 1 uCurie per 96 well. The results showed that tumor-targeted formulation decreased the in vitro IC50 approximately 10-fold. We concluded that tenfibgen nanoparticles were capable of efficiently delivering poorly-soluble small molecule ligands to tumor cells.

### Example 4

### Metal Pretreatment of Coating Ligand Improves Antiproliferative Activity of Nanocapsule Compositions

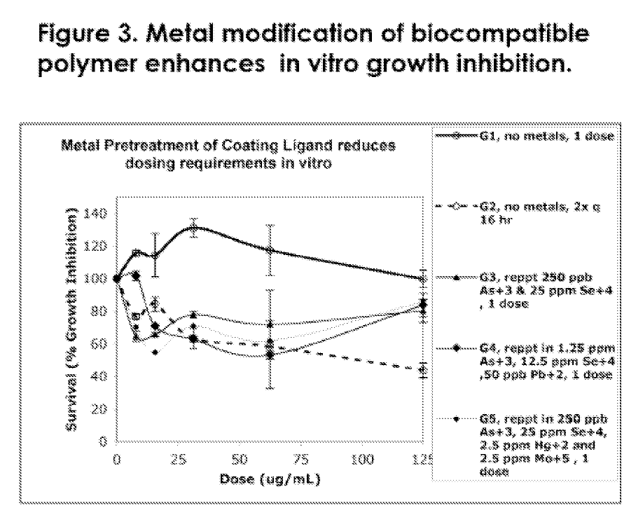

This example shows that biocompatible polymers used in nanoparticles can be pretreated or separately modified with a variety of metal cocktails expected to induce oxidative stress for the purpose of enhancing efficacy of antiproliferative molecules delivered to treatment sites by ligand-targeted nanocapsules. See FIG. 3. The target carcinoma cell line was SCC-15 tongue carcinoma (ATCC CRL-1623) plated on tissue culture plastic at 1500-2000 cells in 96 well plates (SCC-15 retain higher levels of caveolae in culture than many other cell lines, thus not absolutely requiring 3-D cell culture to replace caveolae necessary for particle uptake in this example). Cells were cultured in 10% fetal calf serum in DMEM/F-12 media with pencillin/streptomycin and fungazone. Growth inhibition was measured 72 hours after treatment by the WST (MTT, Roche) assay. The line chart in FIG. 3 shows that nanocapsules prepared from unmodified his-tagged tenfibgen generally as described in Example 1, Formula A, but at a lower coatweight and shorter incubation time (1:80, 14.5 hrs, G1) to promote early dissolution required for short in vitro studies, were unable to inhibit proliferation when delivering the model anti-proliferative molecule, anti-CK2 as 1 dose of 125 ug/ml. However, when in vitro tenfibgen nanocapsules were applied at 2×125 ug/ml doses 24 and 48 hours after plating, significant growth inhibition was achieved (G2). When nanocapsules bearing antisense oligonucleotides were prepared from his-tagged tenfibgen pretreated with various metal cocktails by precipitation from ammonium sulfate (as described elsewhere herein), only 1 dose was required to achieve similar growth inhibition to that achieved by 2 doses of capsules prepared from nonpretreated tenfibgen (G3, G4, G5). As a comparison, at a dose of either 1 or 2×15 ug/ml, survival of treated SCC-15 cells relative to cells treated with diluent is 114±13 or 84±4% for unmodified tenfibgen capsules (1 dose vs. 2 dose) and 65±1, 71±14 or 55% for cells treated with 1 dose of nanoparticles prepared with targeting ligand utilizing the following reprecipitation cocktails (250 ppb As⁺⁴ and 25 ppm Se⁺⁴, 25 ppm As⁺³, 12.5 ppm Se⁺⁴ and 50 ppb Pb⁺² or 250 ppb As⁺³, 25 ppm Se⁺⁴, 2.5 ppm Hg⁺² and 2.5 ppm Mo⁺⁵). We concluded that incorporation of metal ions known to induce oxidative stress into coating ligands preceding particle preparation enhanced overall activity of antiproliferative compositions and that this can be achieved with a variety of compositions comprising metals that will initiate oxidative stress.

### Example 5

### Metal Modification of Nanoparticle Shell Enhances Anti-Tumor Activity of Nucleic Acid Therapeutic In Vivo

This example shows that metal modification of the tenfibgen biocompatible polymer (and resultant co-delivery of ultra-trace levels of metal ions) enhances the efficacy of anti-tumor therapeutic cargos ferried by particles to primary and disseminated disease. Male Scid mice were inoculated subcutaneously in the flank with 5×10e6 of an aggressive head neck carcinoma UM-11A. This slow-growing tumor model is very poorly vascularized, is resistant to treatment by conventional chemotherapy and metastasizes readily from subcutaneous tumors to the lungs and less often to G1. At Day 23 post inoculation, tumors were between 7-9 mm in diameter and mice were randomized into 4 treatment groups. Mice were chronically treated by intraperitoneal administration every three days with either 1) PBS or 10 ug/kg nanoencapsulated (s50) anti-Gapdh (“Controls”, n=3+2), 2) 10 ng/kg s50 anti-CK2 without metal modification (“No Metal”, Formula C, n=3), 3) 10 ng/kg s50 anti-CK2 with metal modification (“10 ng/kg”, Formula D, n=6), and 4) 10 ug/kg s50 anti-CK2 with metal modification (“10 ug/kg”, Formula D, n=6). See Example 1 for formula preparation. Primary tumors were followed by calipher measurement and animal weights were recorded.

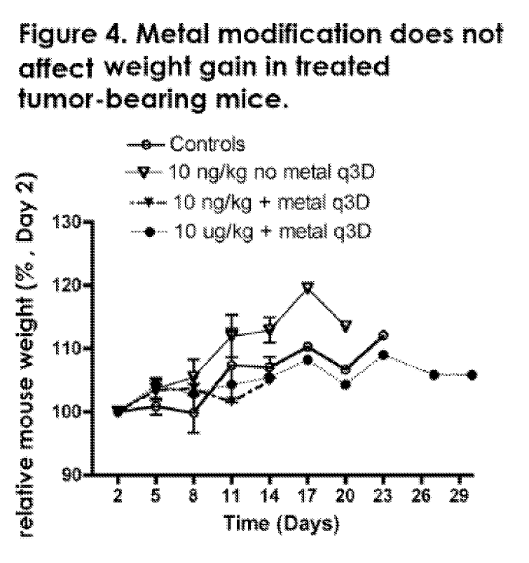

Body weights were followed as a measure of formulation tolerability. See FIG. 4. The line chart shows the running average for treatment group weight over time relative to weight measured on the first day of dosing. As each mouse died, it's contribution to the group average was removed. On an individual basis, all treated mice regardless of treatment showed stable or increasing weight with controls showing the most fluctuation in weight. SSCHN tumors, including xenograft tumors, produce measurable inflammatory cytokines in mice, creating a more difficult situation for weight gain. In addition, no interferon response indicative of an early inflammatory response could be detected in outbred, non-tumor-bearing mice 24 hours after a single 10 mg/kg dose of oligonucleotide formulated with metal-modified biocompatible polymer. We concluded, that surprisingly metal modification did not increase toxicity as would be expected, but instead improved tolerability.

Mice were to have been censored from study when primary tumor reached 15 mm in one dimension. However, only one mouse, an s50 anti-GapDH, was censored for tumor dimension and two mice in the highest treatment group, survived for the duration of the 30 day study. The remaining mice died on study (or were euthanized in respiratory distress) from either lung metastases or lung necrosis as indicated by examination by loupe magnification. Necropsy results are summarized in Table 1 below:

TABLE 1
Necropsy Results from UM-11A study.

| | | | Lung Necrosis | |
|---|---|---|---|---|
| Group | Treatment | Lung Metastases | and/or Scarring | Died on Study |
| 0 | Controls | 5/5 | 0/5 | 4/5 |
| 1 | No Metal | 1/3 | 2/3 | 3/3 |
| 2 | 10 ng/kg | 1/6 | 6/6 | 6/6 |
| 3 | 10 ug/kg* | 0/5 | 4/5 | 4/6 |

*One animal of the original 6 was not recovered for necropsy.

The two survivors from the high dose treatment group showed evidence of previous lung metastases indicating a metastasis rate of 100% in this study. Lung lobes from one survivor contained puckered external scarring in the shape of blood vessels, while in the remaining survivor one of four lobes was about 60% resorbed. Surface vascularization of lung lobes was a striking feature of lung metastasis in this tumor model. These data confirm the capacity of model particle to delivery agents to metastatic disease indicated in Example 2 by killing both disseminated tumor and tumor-derived blood vessels. Inspection of Table 1 indicates that the “No Metal” formulation did show activity against lung metastases (Group 1 vs. Group 0, 1/3 vs. 5/5), but that metastases killing activity was enhanced by metal adsorption of the tenfibgen coating ligand in the composition (Group 2 vs. Group 1, 6/6 vs. 2/3).

Because mice died from lung failure and not primary tumor, the weights of primary tumor at necropsy were small and not different between treatment groups (Controls, 0.3±0.05 g; No Metal, 0.5±0.14 g; 10 ng/kg, 0.3±0.03 g; 10 ug/kg, 0.8±0.4 g). One primary (2.2 g) from one of the survivors was notable in that, while invisible from the surface, it completely covered the ribcage of the animal. Despite the large tumor burden, no active metastases were present in lungs or G1 with continued nanocapsule treatment indicating metal addition improved anti-metastatic activity.

To investigate the effect of metal pretreatment on coating ligand on primary tumor anti-tumor activity we examined vascularity and proliferation state in cryosections by double-label confocal microscopy. Vascularity was quantitated by image analysis in NIH Image J, v. 10.2 as the area fraction of CD 31, CD34 and CD 105 signal intensity in viable tissue areas (antibodies from BD, see Nagatsuka et. al, J Oral Pathol Med (2005) 34:70-6 for validation of microvessel markers in oral cancer). Proliferation state was indexed by quantitation of Ki67 signal area fraction (Chemicon) in viable tissue areas. Signal detection involved thresholding against a control section contained on each slide developed without primary antibodies. Three to four x200 fields of 625×625 microns were captured from two treated sections contained on each slide. Viable areas were defined by Topro and bisbenzamide counterstaining of live nuclei. Results from this quantitative microscopy are summarized in Table 2.

TABLE 2
Analysis of primary tumor sections in UM-11A model.

| | | Vascularity | | | Proliferation | | |
|---|---|---|---|---|---|---|---|
| | | (CD 31, 34, 105, | p (Δ from | p (Δ No | (Ki67 signal, | p (Δ from | p (Δ from |
| Group | Treatment | area percent) | PBS) | Metal) | area percent) | PBS) | No Metal) |
| 0 | Controls | 1.34 ± 0.12 | | | 60.83 ± 8.15 | | |
| 1 | No Metal | 2.14 ± 0.63 | 0.175 | | 51.86 ± 12.21 | 0.467 | |
| 2 | 10 ng/kg* | 2.11 ± 0.19 | 0.136 | 0.953 | 19.23 ± 7.09 | 0.001 | 0.017 |
| 3 | 10 ug/kg | 3.0 ± 0.5 | 0.003 | 0.128 | 15.12 ± 5.22 | 0.001 | 0.008 |

Table displays means ± standard Errors. Significance of difference between means was calculated by ANOVA with post hoc Fisher's testing
*One tumor sample was lost so that n = 5 for this analysis.

Inspection of Table 2 shows that, overall, nanocapsule treatment appears to have increased the vascularity of these poorly vascular tumors and decreased the proliferation state of viable tumor cells. With respect to vascularity, this increase was an ˜2× trend for both metal and unmodified dosing at 10 ng/kg but a significant ˜3× increase at 10 ug/kg. In treated tumors, blood vessels were observed near necrotic regions indicating a wound response following anti-tumor activity. Normalization of tumor vascularity in a poorly vascular tumor predicts positive combination with conventional chemotherapy agents which depend on tumor vasculature for tumor access and constitutes anti-tumor activity. With respect to proliferation state, metal modification of coating ligand significantly decreased (0.37× or ±63%) proliferation state of viable tumor cells at 10 ng/kg dosing. Proliferation state was not decreased further by higher dosing. We concluded metal modification of coating ligand significantly enhanced anti-tumor activity of nanoencapsulated anti-tumor compounds, while also enabling lower overall dosing. We also concluded that due to the increase in anti-tumor activity following metals addition together with the anti-metastatic activity observed at necropsy that nanoparticles cellular targeting was not compromised by metal modification of the targeting ligand.

These results are altogether surprising due to the ultra-trace loading of metal in these compositions. If we consider that the targeting ligand is only 4.7% by weight of the formulation and assuming even a 50% complexing from the precipitation step, the metal load in one ug of formulation would be ({0.25+25+2.5+25 or 52.75 ppm}*0.5*lug*0.047) or 1.24 picograms. This translates to a repeat dose of metals of 161 femtograms for a 20 gram mouse at a dose of 10 ug/kg of s50 oligonucleotide. These results also indicate that an unexpected synergy occurs at low dosing when combining particle shell metal-modification and a therapeutic cargo as the unmodified therapeutic particle has no effect on primary tumor at low dosing and metal-modified particles bearing non-therapeutic loads (e.g. sugar, anti-reporter genes, etc.) have no anti-tumor activity across several models at even higher dosing levels.

### Example 6

### Treatment of Mice Xenograft Cancer Model with Metal-Enhanced Small Molecule Nanocapsules

This example shows that metal-modified nanoparticles are useful for in vivo delivery of anti-tumor small molecule cargos to disseminated disease. A small molecule, Paclitaxel (to be prepared as in Example 1, Formula E, with average diameter being found to be about 5-15 nanometers) is administered to nude mice engrafted with 4 million Fadu SSCHN hypopharangeal carcinoma cells on the flank (ATCC HTB-43). This tumor metastasizes to the lungs almost immediately upon flank inoculation. Mice are treated intravenously when tumors are approximately 4 mm in diameter. The doses range from 10 pg/kg to 10 ug/kg in chronic administered 3 days apart. Tumors are measured using calipers 1-2× per week and mice held on survival for 3 months. Mice survival is compared to diluent and treatment with unformulated species. Demonstration of increased survival relative to controls shows that metal-modified particles enhance efficacy and decrease dosing requirements.

### Example 7

### Treatment of Mice Xenograft Cancer Model with Metal-Enhanced Oligonucleotide Nanocapsules

This example shows that metal-modified hyaluronan nanoparticles are useful for in vivo delivery of anti-tumor therapeutic cargos to disseminated disease. An antisense oligo directed against both BCL-xL and BCL-2 (to be prepared as in Example 1, Formula F, with average diameter being found to be about 15 nanometers) is administered to nude mice engrafted with 4 million Fadu SSCHN hypopharangeal carcinoma cells on the flank (ATCC HTB-43). This tumor metastasizes to the lungs almost immediately upon flank inoculation. Mice are treated intravenously when tumors are approximately 6 mm in diameter. The doses range from 10 ng/kg to 1 mg/kg in chronic dosing administered 3 days apart. Tumors are measured using calipers 1-2× per week and mice held on survival for 3 months. Mice survival is compared to diluent and treatment with unformulated species. Demonstration of increased survival relative to controls shows that metal-modified particles enhance efficacy and decrease dosing requirements.

It is to be understood that while the invention has been described in conjunction with the detailed description thereof, the foregoing description is intended to illustrate and not limit the scope of the invention, which is defined by the scope of the appended claims. Other aspects, claims, advantages and modifications are within the scope of the following claims.

1. A method for forming particles useful for the treatment of hyperproliferative disease, the method comprising:
providing a bioactive component;
providing a metal ion-treated biocompatible polymer component;
coating the bioactive component with a surfactant having an HLB value of less than about 6.0 units under conditions which form a coated bioactive component;
associating the coated bioactive component with a metal ion-treated biocompatible polymer under conditions which associate the coated bioactive component with the metal-ion treated biocompatible polymer to form a particle,
wherein the particles have an average diameter of less than about 50 nanometers as measured by atomic force microscopy of the particles following drying of the particles.

2. The method of claim 1, wherein the hyperproliferative disease is cancer.

3. The method of claim 1, wherein the metal ion-treated biocompatible polymer component is treated by the following steps:
providing a biocompatible polymer capable of being precipitated;
combining the biocompatible polymer, a metal ion solution, and a precipitant;
precipitating the metal ion-treated biocompatible polymer; and
resolubilizing the metal ion-treated biocompatible polymer, forming a metal ion-treated biocompatible polymer component.

4. The method of claim 3, wherein the biocompatible polymer component is a polypeptide.

5. The method of claim 3, wherein the biocompatible polymer component is a carbohydrate.

6. (canceled)

7. The method of claim 3, wherein the metal ion solutions comprise at least one metal ion which promotes oxidative stress.

8. The method of claim 3, wherein the metal ion solution comprises at least one cation selected from the group consisting of arsenic cations, selenium cations, molybdenum cations, mercury cations, and combinations thereof.

9. The method of claim 8, wherein the metal ion solution comprises the at least one cation in a concentration of between about 0.1 part per billion (ppb) and 1 part per thousand (ppt) and wherein the metal ion solution comprises a total metal ion concentration not exceeding 10 parts per thousand.

10. The method of claim 9, wherein the metal ion solution comprises a total metal ion concentration not exceeding 2 parts per thousand.

11. The method of claim 1, wherein the bioactive component is a nucleic acid.

12. The method of claim 1, wherein the bioactive component is a pharmaceutically-active small molecule.

13. The method of claim 1, wherein the step of associating the coated bioactive component with the metal ion-treated biocompatible polymer comprises adding the coated bioactive component with the metal ion-treated biocompatible polymer in aqueous solution.

14. (canceled)

15. A method for preparing a metal-treated biocompatible polymer composition, comprising:
providing a biocompatible polymer capable of being precipitated;
combining the biocompatible polymer, a metal ion solution, and a precipitant;
precipitating the metal ion-treated biocompatible polymer; and
resolubilizing the metal ion-treated biocompatible polymer, thereby forming a metal ion-treated biocompatible polymer composition.

16. The method of claim 15, further comprising incorporating the metal ion-treated biocompatible polymer into a pharmaceutical formulation.

17. A composition of particles comprising:
(a) a bioactive component;
(b) a surfactant having an HLB value of less than about 6.0 units, said surfactant being associated with the bioactive component; and
(c) a metal ion-treated biocompatible polymer surrounding the association of the bioactive component and said surfactant, wherein at least one of said biocompatible polymers provides specific cellular or tissue uptake,
wherein the particles have an average diameter of less than about 50 nanometers as measured by atomic force microscopy following drying of the particles.

18. The composition of claim 17 wherein the macromolecule comprises a polynucleic acid, oligonucleotide, antisense molecule, or peptide nucleic acid.

19. The composition of claim 17, wherein the metal ion-treated biocompatible polymer component is a polypeptide.

20. The composition of claim 17, wherein the metal ion-treated biocompatible polymer component is carbohydrate.

21. The composition of claim 17, wherein the metal ion comprises at least one cation selected from the group consisting of arsenic cations, selenium cations, molybdenum cations, mercury cations, and combinations thereof.

22. The composition of claim 21, wherein the at least one cation comprises less than about 1.2 picograms per microgram of particle therapeutic.

23. (canceled)
